(19) United States
(12) Patent Application Publication
(10) Pub. No.: US 2023/0094837 A1
(43) Pub. Date: Mar. 30, 2023

(54) Biodegradable and Multifunctional Neural Block Devices

(71) Applicant: The Penn State Research Foundation, University Park, PA (US)

(72) Inventors: Jian Yang, State College, PA (US); Ethan Gerhard, State College, PA (US); Yuqi Wang, State College, PA (US); Huanyu Cheng, University Park, PA (US); Sanjib Das Adhikary, Hershey, PA (US)

(21) Appl. No.: 17/758,539

(22) PCT Filed: Apr. 7, 2021
(86) PCT No.: PCT/US21/26137

§ 371 Date: Jul. 8, 2022

Related U.S. Application Data
(60) Provisional application No. 63/006,521, filed on Apr. 7, 2020.

(51) Int. Cl. A61L 29/06; A61M 25/00; A61N 1/36; A61M 25/04; A61L 29/14; A61L 29/16

(52) U.S. Cl. CPC A61L 29/06; A61M 25/0026; A61N 1/36071; A61M 25/04; A61L 29/148; A61L 29/16; A61M 25/0043; A61M 2025/0057; A61M 2205/0266; A61M 2205/8262; A61L 2300/204; A61L 2300/402

(57) ABSTRACT

Embodiments relate to a crosslinked citrate-based elastomer catheter that is biodegradable and kink resistant. Embodiments of the crosslinked citrate-based elastomer material swells when surrounded by fluid (body fluid) so as to anchor the catheter to tissue but not anchor it so much that movement or removal will cause tissue damage. The catheter can be used as a component to a peripheral nerve block device, for example. Embodiments of the catheter can include embedding biodegradable sensors, moieties, shape memory material, etc. to monitor and modulate functions of the catheter and/or peripheral nerve block.

### CROSS-REFERENCE TO RELATED APPLICATIONS

This application is related to and claims the benefit of priority of U.S. provisional application 63/006,521, filed Apr. 7, 2020, the entire contents of which is incorporated herein by reference.

### FIELD OF THE INVENTION

Embodiments relate to a crosslinked citrate-based elastomer catheter that is biodegradable and kink resistant. Embodiments of the crosslinked citrate-based elastomer material swells when surrounded by fluid (body fluid) so as to anchor the catheter to tissue but not anchor it so much that movement or removal will cause tissue damage.

### BACKGROUND OF THE INVENTION

Surgical procedures can require effective methods of peri- and post-operative pain management in order to ensure patient recovery and satisfaction. In contrast to systemic and epidural anesthetic procedures, peripheral nerve block (PNB) offers specific, localized effects, reducing consumption of potentially addictive opioids. PNBs involve introduction of drugs and other agents via a catheter to a local site—the site at which surgery is being performed. Particularly, continuous PNBs have shown a marked capability to reduce hospitalization duration and concomitant costs, minimize re-hospitalization events, increase physical therapy compliance, and alleviate concerns of local neural toxicity by reducing drug concentration. However, despite the evident advantages, less than one in five procedures utilizes this technique, as a result of serious complications arising from the extended dwell time and necessary removal of the nondegradable catheters used for the PNB devices. These complications include inflammation, bleeding, tissue damage, fibrous encapsulation, tube kinking, tube knotting, tube looping, tube fracture, tube displacement, difficult catheter removal, vascular puncture/hematoma, nerve puncture/damage, etc.

Conventional PNB catheters are composed of non-degradable materials such as polyurethane, polyamide, and stainless steel. While such materials are desirable for their strength and durability (as well as electrical conductivity in the case of metals), they elicit negative tissue responses including inflammation (13.7%) and bacterial infection (7.5-57%) as well as tissue adhesion and foreign body encapsulation. Adverse implant/tissue interactions can lead to difficulty in catheter removal. In extreme cases, surgical intervention is required. To address this, conventional PNB catheter systems focus heavily on avoidance of tissue adhesion, but this leads to a problem of catheter dislodgement. Catheter dislodgement can result in ineffective drug delivery and potential tissue damage.

Conventional PNB and catheter devices and methods of use can be appreciated from:
D. I. Mclsaac, C. J. McCartney, C. V. Walraven, Peripheral Nerve Blockade for Primary Total Knee Arthroplasty: A Population-based Cohort Study of Outcomes and Resource Utilization, Anesthesiology 126(2) (2017) 312-320.
E. M. Soffin, J. T. YaDeau, Peripheral Nerve Catheters: Ready for a Central Role?, Anesth Analg 124(1) (2017) 4-6.
G. Joshi, K. Gandhi, N. Shah, J. Gadsden, S. L. Corman, Peripheral nerve blocks in the management of postoperative pain: challenges and opportunities, J Clin Anesth 35 (2016) 524-529.
T. T. Horlocker, S. L. Kopp, M. W. Pagnano, J. R. Hebl, Analgesia for total hip and knee arthroplasty: a multimodal pathway featuring peripheral nerve block, J Am Acad Orthop Surg 14(3) (2006) 126-35.
Z. S. Ahsan, B. Carvalho, J. Yao, Incidence of failure of continuous peripheral nerve catheters for postoperative analgesia in upper extremity surgery, J Hand Surg Am 39(2) (2014) 324-9.
C. L. Jeng, T. M. Torrillo, M. A. Rosenblatt, Complications of peripheral nerve blocks, Br J Anaesth 105 Suppl 1 (2010) i97-107.
D. Simic, M. Stevic, Z. Stankovic, I. Simic, S. Ducic, I. Petrov, M. Milenovic, The Safety and Efficacy of the Continuous Peripheral Nerve Block in Postoperative Analgesia of Pediatric Patients, Front Med (Lausanne) 5 (2018) 57.
S. D. Adhikary, K. Armstrong, K. J. Chin, Perineural entrapment of an interscalene stimulating catheter, Anaesth Intens Care 40(3) (2012) 527-530.
S. R. Clendenen, C. B. Robards, R. A. Greengrass, S. J. Brull, Complications of peripheral nerve catheter removal at home: case series of five ambulatory interscalene blocks, Can J Anesth 58(1) (2011) 62-67.
R. Duclas, C. B. Robards, B. L. Ladie, S. R. Clendenen, Tip adhesions complicate infraclavicular catheter removal, Can J Anesth 58(5) (2011) 482-483.
G. B. Kim, Y. Chen, W. Kang, J. Guo, R. Payne, H. Li, Q. Wei, J. Baker, C. Dong, S. Zhang, P. K. Wong, E. B. Rizk, J. Yan, J. Yang, The critical chemical and mechanical regulation of folic acid on neural engineering, Biomaterials 178 (2018) 504-516.
J. M. Neal, Effects of epinephrine in local anesthetics on the central and peripheral nervous systems: Neurotoxicity and neural blood flow, Region Anesth Pain M 28(2) (2003) 124-134.
M. Mehdizadeh, H. Weng, D. Gyawali, L. P. Tang, J. Yang, Injectable citrate-based musselinspired tissue bioadhesives with high wet strength for sutureless wound closure, Biomaterials 33(32) (2012) 7972-7983.
M. Damjanovska, E. Cvetko, M. M. Kuroda, A. Seliskar, T. Plavec, K. Mis, M. Podbregar, T. S. Pintaric, Neurotoxicity of intraneural injection of bupivacaine liposome injectable suspension versus bupivacaine hydrochloride in a porcine model, Vet Anaesth Analg 46(2) (2019) 236-245.
R. T. Tran, P. Thevenot, D. Gyawali, J. C. Chiao, L. P. Tang, J. Yang, Synthesis and characterization of a biodegradable elastomer featuring a dual crosslinking mechanism, Soft Matter 6(11) (2010) 2449-2461.
B. Y. Zhang, M. Montgomery, L. Davenport-Huyer, A. Korolj, M. Radisic, Platform technology for scalable assembly of instantaneously functional mosaic tissues, Sci Adv 1(7) (2015).

J. S. Guo, W. Wang, J. Q. Hu, D. H. Xie, E. Gerhard, M. Nisic, D. Y. Shan, G. Y. Qian, S. Y. Zheng, J. Yang, Synthesis and characterization of anti-bacterial and anti-fungal citrate-based mussel inspired bioadhesives, Biomaterials 85 (2016) 204-217.
D. Shan, C. Ma, J. Yang, Enabling biodegradable functional biomaterials for the management of neurological disorders, Adv Drug Deliv Rev (2019).
D. Y. Shan, S. R. Kothapalli, D. J. Ravnic, E. Gerhard, J. P. Kim, J. S. Guo, C. Y. Ma, J. Z. Guo, L. Gui, L. Sun, D. Lu, J. Yang, Development of Citrate-Based Dual-Imaging Enabled Biodegradable Electroactive Polymers, Adv Funct Mater 28(34) (2018).

### BRIEF SUMMARY OF THE INVENTION

Embodiments relate to a catheter having a versatile and biocompatible citrate-based material platform. Material optimization through the molecular, micro, and macro levels provide for a fully biodegradable, tissue adherent peripheral nerve catheter capable of sustained drug delivery without damage to surrounding tissue. The catheter has high strength, is elastic, and is kink resistant. The device is a rapidly degradable biphasic catheter capable of promoting tissue adherence sans aggressive immune response and eliminating the need for removal, thus solving the conflicting need to attain device security during treatment and detachment post-treatment.

In an exemplary embodiment, a catheter includes an elongated body defining one or more lumen. The elongated body is composed of a biodegradable crosslinked polymer.

In some embodiments, the crosslinked polymer is citrate/xylitol-based elastomer (CXBE).

Some embodiments include a monitoring system comprising at least one moiety embedded within the elongated body, the moiety configured as a sensor.

Some embodiments include a delivery system comprising at least one moiety embedded within the elongated body, the moiety configured to controllably release an agent encapsulated within the moiety.

Some embodiments include a modulation system comprising at least one moiety embedded within the elongated body, the moiety configured to modulate flow of an agent through the one or more lumen or a portion of the elongated body.

Some embodiments include a modulation system comprising at least one sensor embedded within the elongated body, a delivery system comprising an encapsulation material that releases an agent upon degradation of the encapsulation or a shape memory material that releases an agent upon being activated, and a modulation system that controls the degradation of the encapsulation material or controls the activation of the shape memory material.

Some embodiments include a power harvester in electrical connection with any one or combination of: a modulation system comprising at least one sensor embedded within the elongated body; a delivery system comprising an encapsulation material that releases an agent upon degradation of the encapsulation or a shape memory material that releases an agent upon being activated; and a modulation system that controls the degradation of the encapsulation material or controls the activation of the shape memory material.

Some embodiments include a wireless module in electrical connection with the power harvester and in wireless communication with an operating module.

Some embodiments include a pair of electrodes configured to generate electrical stimuli.

Some embodiments include an anchoring mechanism configured to anchor the catheter to tissue.

In some embodiments, the anchoring mechanism includes any one or combination of: surface roughness of the catheter; nano- or micro-structures formed on a surface of the catheter; porous structures formed on a surface of the catheter; or adhesion moieties formed on a surface of the catheter.

In some embodiments, the CXBE is incorporated with epinephrine to generate epinephrine bearing CXBE (eCXBE).

In some embodiments, lidocaine is encapsulated within the eCBXE to generate eCBXE/lidocaine.

In some embodiments, the biodegradable crosslinked polymer contains a fluorescent polymer.

In some embodiments, the biodegradable crosslinked polymer has a differentiated crosslinked density through a cross-sectional portion of the elongated body. The differentiated crosslinked density leads to differentiated swelling of the elongated body during water uptake.

In an exemplary embodiment, a method of administering peripheral nerve block involves: inserting a catheter in tissue of a patient; allowing the catheter to swell so as to cause catheter anchorage to the tissue; delivering agent via the catheter; and allowing the catheter to biodegrade.

In some embodiments, swelling is the only form of tissue anchorage for the catheter.

In some embodiments, the catheter includes an elongated body defining one or more lumen. Swelling at or near the one or more lumen is less than swelling at an outer periphery of the elongated body.

Some embodiments involve monitoring functionality and material degradation of the catheter via at least one moiety embedded within the catheter.

Some embodiments involve delivering agent via at least one moiety embedded within the catheter.

Some embodiments involve modulating flow of agent via at least one moiety embedded within the catheter.

Some embodiments involve: monitoring functionality and material degradation of the catheter via at least one monitoring moiety embedded within the catheter; delivering agent via at least one delivering moiety embedded within the catheter; modulating flow of agent via at least one modulating moiety embedded within the catheter; and providing electrical power to any one or combination of the monitoring moiety, the delivering moiety, or the modulating moiety via a power harvester embedded within the catheter.

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages, and possible applications of the present invention will be more apparent from the following more particular description thereof, presented in conjunction with the following drawings. It should be understood that like reference numbers used in the drawings may identify like components.

FIG. 1 shows an exemplary peripheral nerve block device and catheter system.

FIG. 2 shows an exemplary catheter having a monitoring, delivery, and modulation system incorporated therein.

FIG. 3 shows the general synthesis of CXBE elastomers.

FIG. 4 shows an exemplary biphasic CXBE catheter fabrication process.

FIG. 5 shows an exemplary fabrication procedure for generating a CXBE catheter with a biomimetic anchor design.

FIG. 6 shows a fabrication method that incorporates epinephrine into CXBEs through an esterification reaction to generate epinephrine bearing (eCXBE) polymers.

FIG. 7 shows a fluorescent spectrum of X6 crosslinked 3D-80C, 3D-120C (inset shows maximum emission spectrum of X6, P1, and P2 crosslinked 3D-80C, 3D-120C).

FIG. 8 shows ultraviolet and daylight images of films and catheter tubes.

FIG. 9 shows fluorescent images of X6, P1, and P2 under various excitation wavelengths.

FIG. 10 shows fluorescence spectrum of reaction products of citric acid and lidocaine (inset shows CA-Lidocaine solution in DI water under daylight and UV illumination).

FIG. 11 shows fluorescence spectrum of X6-L20 crosslinked 3D-80C (inset shows spectra of X6-L20, P1-L20, and P2-L20).

FIG. 12 shows chemical structures of ester- and amide-type anesthetics.

FIG. 13 shows a reaction mechanism of citric acid and lidocaine.

FIG. 14 shows representative ester-type anesthetics.

FIG. 15 shows representative amide-type anesthetics.

FIGS. 16 and 17 show tensile stresses for CXBE films.

FIG. 18 shows initial moduli for PEG containing CXBEs.

FIG. 19 shows strains of PEG containing CXBEs.

FIG. 20 shows molecular weights and polydispersity indexes of CXBE prepolymers.

FIG. 21 shows density of synthesized CXBEs.

FIG. 22 shows molecular weight between crosslinks of synthesized CXBEs.

FIG. 23 shows CXBE catheters displaying enhanced kink resistance.

FIG. 24 shows a conventional polyurethane catheter exhibiting poor kink resistance.

FIG. 25 shows insertion of a CXBE catheter into cadaver tissue, CXBE catheter implanted, and ultrasound guidance of CXBE catheter (catheter circled).

FIG. 26 shows an intact CXBE catheter post-insertion (catheter circled).

FIG. 27 shows a fabrication of a biphasic catheter comprising inner solid inner phase and outer porous segment capable of tissue infiltration and anchorage.

FIG. 28 shows a detailed fabrication procedure for biphasic porous catheters.

FIG. 29 shows a fabrication process for a single phase lidocaine releasing catheter.

FIG. 30 shows a fabrication process for a catheter containing lidocaine in the proximal region.

FIG. 31 shows fabrication of a catheter with outer lidocaine releasing phase and inner drug free phase.

FIG. 32 shows: (A) Fluorescence and (B) Digital image of Zn sensors deposited on CXBE films, (C) Fluorescence and (D) Digital image of Zn sensor deposited on a CXBE catheter.

FIG. 33 shows deposition of conductive Zn layer of CXBE films.

FIG. 34 shows voltage vs. current graph of Zn sensor deposited on film showing conductivity.

FIG. 35 shows voltage vs. current graph of Zn sensor deposited on catheter showing conductivity.

FIG. 36 shows piezoelectric and electrostimulation functions of CXBE catheters with Zn sensor.

FIG. 37 shows a brightfield microscope image of catheter cross-section (scale bar 500 um).

FIG. 38 shows a horizontal microscope image of catheter with defined lumen and wall regions.

FIG. 39 shows a magnified view of a catheter wall.

FIG. 40 shows radial force required to compress X6 and commercial catheters to 50% initial lumen diameter.

FIG. 41 shows a compression fixture utilized to test radial force.

FIG. 42 shows swelling of CXBE films following 24 hr immersion in PBS at 37C.

FIG. 43 shows volume swelling of X6 disks following 24 hr immersion decreased swelling with increased crosslinking time.

FIG. 44 shows a diagram depicting tissue anchorage mechanism utilizing differential swelling of inner and outer catheter layers following implantation.

FIG. 45 shows volume swelling of disks following 7 days of immersion in PBS showing rapid degradation of P2.

FIG. 46 is a schematic showing the synthesis of representative xylitol doped poly(octamethylene citrate).

FIG. 47 shows the density of representative polymers of the disclosure as synthesized in the examples. The data demonstrates an increase in density with increased xylitol content within the polymer.

FIG. 48 shows the measured molecular weight of the crosslinks in representative polymers of the disclosure as synthesized in the examples. Polymers containing xylitol were found to have a highly crosslinked structure as compared to conventional POC, leading to enhanced mechanical properties.

FIG. 49 shows the Fourier-transform infrared spectrogram for representative polymers of the disclosure as synthesized in the examples. An increased —OH signal was found with increased xylitol content, indicating the formation of hydrogen bonding between polymer chains which further reinforces polymer mechanics.

FIG. 50 are x-ray diffraction spectra for representative polymers of the disclosure as synthesized in the examples. The spectra depict a lack of crystallinity of the polymers induced by increase xylitol content.

FIGS. 51A, 51B, 51C, 51D, 51E, 51F, and 51G show tensile film mechanics for films formed from representative polymers of the present disclosure as described in the examples. These measurements demonstrate the tunability of film mechanics in a manner that is capable of matching a range of biological tissues such as skin, nerve, bone, etc.

FIGS. 52A and 52B show the measured external contact angle for representative polymers of the present disclosure as described in the examples. These data show the hydrophilicity of the representative materials.

FIG. 53 provides data showing enhanced fluorescence of the representative polymers with increasing xylitol content.

FIGS. 54A, 54B, 54C, 54D, 54E, 54F, and 54G show fluorescence emission spectra for representative polymers of the present disclosure. These spectra show that the disclosed compositions are capable of imaging and light delivery in vivo.

FIG. 55 shows measurements of compressive stress for representative compositions of the disclosure further comprising 60 weight percent hydroxyapatitite (HA). These data demonstrate uniform stress on the compositions regardless of xylitol content.

FIG. 56 shows measurements of compressive modulus for representative formulations of the disclosure further comprising 60 weight percent hydroxyapatite. These measurements are significantly equivalent compared to composites lacking xylitol as a monomer component.

FIG. 57 shows measurements of compressive strain for representative compositions further comprising 60 weight percent hydroxyapatite (HA).

FIG. 58 shows the percentage of swell for representative compositions of the present disclosure. The data show that composites containing xylitol swell at the same rate as composites lacking xylitol despite the increased hydrophilic character of said monomer component.

FIG. 59 shows the percent degradative loss of representative compositions over time. Degradation was found to be tunable from 5% to 40% (i.e., complete degradation of the polymer component) over a 16 week period. When viewed in combination with the associated mechanical data for the representative polymers, these data demonstrate wide tunability of composition degradation without any negative impact on mechanics of the composition.

FIG. 60 shows measurements of pH versus time for representative compositions of the disclosure. These data show a return to ˜7.4 pH (physiological) within one week. Therefore, the compositions of the present disclosure are capable of replicating a desired pH profile.

FIGS. 61A and 61B show fluorescence and room temperature phosphorescence, respectively, for compositions of the disclosure containing hydroxyapatite (POCX6/50HA). These demonstrate that the disclosed compositions may be used with multiple imaging modalities. In particular, phosphorescence may be preferred for imaging in vivo to avoid the autofluorescence of biological tissue through the intrinsic delayed emission of phosphorescence versus fluorescence.

FIGS. 62A, 62B, and 62C show in vitro cytotoxicity evaluation against MG63 cell of the degradation products for disclosed compositions as described in the examples as well as the cytotoxicity of leachable components and degradation products for such compositions further comprising hydroxyapatite (CXBE/50HA).

### DETAILED DESCRIPTION OF THE INVENTION

The following description is of an embodiment presently contemplated for carrying out the present invention. This description is not to be taken in a limiting sense but is made merely for the purpose of describing the general principles and features of the present invention. The scope of the present invention should be determined with reference to the claims.

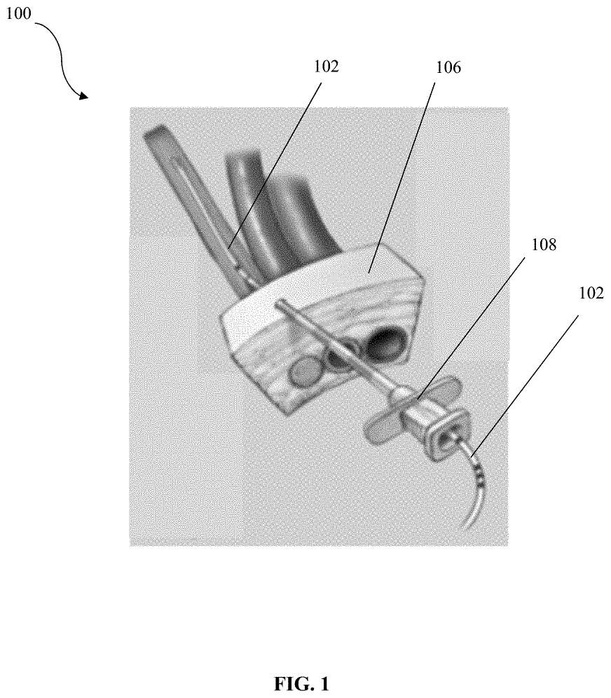

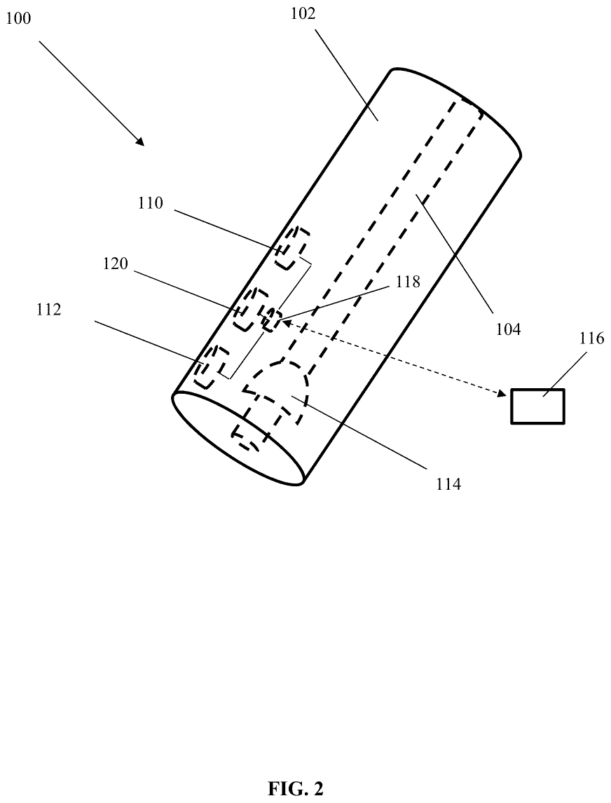

Referring to FIGS. 1-2, embodiments relate to a crosslinked citrate-based elastomer catheter 102 that is biodegradable and kink resistant. The catheter 102 is an elongated tubular structure having one or more lumen 104 formed therein. The lumen 104 runs along a longitudinal axis of the catheter 102 to serve as a conduit for fluid transmission (e.g., transmission of liquids, gels, particles, solids, other agents, or combinations thereof). An exemplary use of a catheter 102 is to insert one end (e.g., the distal end) into the body of a patient and connect the other end (e.g., the proximal end) to a fluid delivery or fluid retrieval device. Some embodiments of the catheter 102 have more than one lumen 104. The multiple lumen 104 can be used to supply different agents via the different lumen 104, supply fluid via one lumen 104, draw fluid from another lumen 104, etc. The catheter 102 can be structured to deliver agents over a period of between 1 day and 1 month, for example. It is important for the catheter 102 material to exhibit the mechanical strength to be kink resistant, but it is also highly desirable for the catheter 102 material to be biodegradable. The crosslinked citrate-based elastomer disclosed herein achieves these combined properties. For instance, it is contemplated for embodiments of the catheter 102 to have the mechanical structure to facilitate bending, twisting, and knotting without suffering a mechanical failure, rupture, kinking, or lumen 104 collapse, while also being able to biodegrade completely within one month to one year. In addition, embodiments of the crosslinked citrate-based elastomer material swells when surrounded by fluid (body fluid) so as to anchor the catheter 102 to tissue 106 but not anchor it so much that movement or removal will cause tissue 106 damage.

The catheter 102 can be used as a component to a peripheral nerve block device (PNB) 100, for example. A PNB device 100 is a device that facilitates administration of a regional anesthesia—e.g., allowing for injection of anesthetic at or near a specific nerve or bundle of nerves. The anesthesia can be a drug (e.g., analgesic) delivered via a catheter 102 to a local site (the site being the nerve or nerve bundle). For instance, the PNB device 100 can be used to deliver anesthetic/analgesic compounds for the purposes of performing surgery and pain management post-surgery. Such compounds may be delivered as single injections or sustained infusions over periods ranging from several hours to two months. Thus, the PNB device 100 can be the catheter 102. Some embodiments of the PNB device 100 might include a trocar 108 to assist with guidance, placement, and retention of the catheter 102. As will be explained herein, embodiments of the PNB device 100 can include monitoring 110, delivery 112, and/or modulation 114 systems. These can include biodegradable sensors, moieties, shape memory material, power harvesting devices, etc. to monitor and modulate functions of the catheter 102 and/or PNB device 100. For instance, an exemplary monitoring system 110 can be a biodegradable sensor embedded within the catheter 102 configured to measure temperature, pressure, blood oxygen, pH level, etc. An exemplary delivery system 112 can be a biodegradable shape memory material embedded within the catheter 102 configured to controllably release a drug under certain conditions. An exemplary modulation system 114 can be a biodegradable shape memory material embedded within the catheter 102 and disposed about a periphery of any one or combination of lumen 104 within the catheter 102 so as to controllably constrict or dilate the lumen 104.

While embodiments may illustrate a catheter 102 system in which the catheter 102 is inserted through a needle, embodiments of the material can be used for catheter 102 systems in which the catheter 102 is slid over the needle.

Biodegradable materials offer an effective alternative to conventionally used catheter 102 materials, with the capability to be strongly secured during the treatment period (e.g., one to two weeks weeks) without the need for removal post-treatment. Citrate, well known for its canonical role in the metabolic cycle, can be used as a multifunctional monomer for polymer synthesis. Through a series of simple catalyst-free reactions, crosslinked citrate-based elastomers (CBEs), typified by poly(octamethylene citrate) (POC) can be synthesized. POC, as well as its constituent monomers, citric acid and octanediol, are biocompatible both in vitro and in vivo, with excellent blood and tissue compatibility, and minimal inflammatory response. POC is also highly tunable through modification both during and post-polymerization, resulting in a wide range of materials including biodegradable photoluminescent polymers (BPLPs), injectable poly(alkylene maleate citrate) (PAMC), and mussel inspired bioadhesives (iCMBA). CBEs present tunable properties including mechanics, degradation rates (from days to >one year), and swelling ratios. CBEs are also readily fabricated into films, scaffolds, and tubes. Notably, for applications involving catheters 102 and PNB devices 100, CBEs are effective in vivo as flexible, kink-resistant nerve regeneration guides. CBE PNB catheters 102 also exhibit compatibility with neural tissue 106. However, CBE by itself still suffers from low mechanical strength and long degradation times (e.g., twenty six weeks for POC). It is possible to increase mechanical properties of CBE through the incorporation of click chemistry (POC-Click) or urethane doping (crosslinked urethane doped polyesters, CUPEs), but this results in an unacceptable decrease in biodegradation rate.

With a goal of providing a biomaterial capable of both high mechanical strengths and rapid degradation, the inventors focused on polymer network engineering. High strength crosslinked CBE materials can be achieved through maximization of crosslink density; however, it was found that use of linear monomers such as diols, diisocyanates, etc. limits the maximum achievable crosslink density. Thus, polyfunctional alternatives were considered. With polyfunctional alternatives, the resultant increase in crosslink density is typically countered by a large decrease in biodegradation rate unless careful selection of the desired monomer is used.

Xylitol, a five-carbon pentinol, can be used as a crosslink density enhancer due to its five reactive hydroxyl groups liable to polycondensation with citrate. Additionally, xylitol is a hydrophilic, water-soluble monomer, capable of enhancing water uptake and thus hydrolysis of ester bonds in CBE networks, increasing degradation. Additionally, increased water uptake results in significant swelling and expansion upon contact with body fluids. This swelling and expansion results in sufficient anchoring of the material in the surrounding tissue. Additionally, xylitol is a nontoxic sugar alcohol routinely used as a sugar alternative and in oral rinses. Thus, the disclosed citrate/xylitol-based elastomer (C)CBE) can be engineered to achieve high crosslink density, combining desirable properties of vastly increased mechanical properties, accelerated degradation, and increased swelling based anchoring.

Some embodiments can incorporate a secondary cross-linking mechanism, such as but not limited to isocyanate cross-linking, ultraviolet or redox-mediated free radical cross-linking, click chemistry, Schiff base formation, thiol-ene, Michael Addition, or Diels Alder chemistry with primary thermal cross-linking.

It is contemplated for polyisocyanate macromer or polymer to be used to set citrate-based materials during preparation of the CXBE. Such a polyisocyanate could also be a functionalized ceramic or other additives. Some embodiments include combining a citrate-based polymer with a component that acts both as a solvent for said citrate-based polymer but also as a polyfunctional reactant.

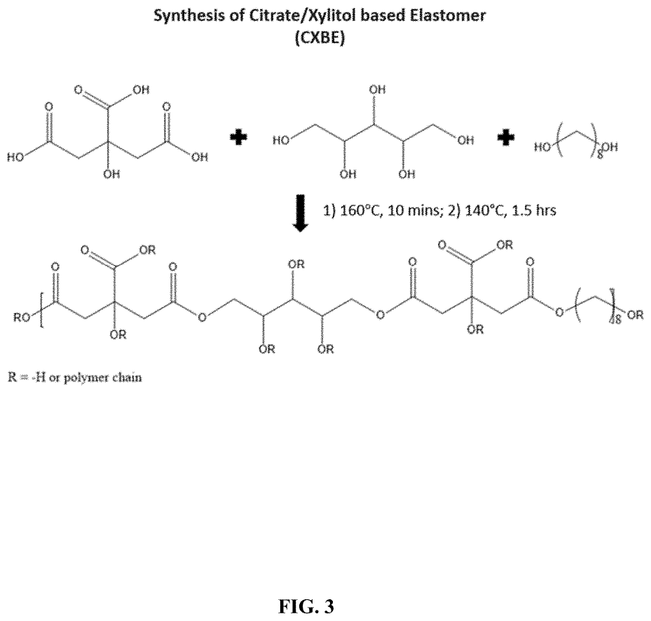

Referring to FIG. 3, in an exemplary embodiment, CXBE pre-polymer is synthesized via a one-pot polycondensation reaction. With this exemplary synthesis, citric acid, xylitol, and octanediol with a 1:1 mole ratio of citric acid:(octanediol and xylitol) are melted at 160° C. under stirring for ten minutes. The reaction temperature is then reduced to 140° C. The reaction proceeds until the pre-polymer can no longer be stirred due to viscosity, at which point the reaction is quenched with dioxane. Referring to Table 1, CXBE prepolymers may be synthesized with a variety of mol ratios.

TABLE 1
Molar Ratios of Various CXBE Formulations

| | Citric Acid | Xylitol | Octanediol |
|---|---|---|---|
| | (mols) | (mols) | (mols) |
| POC | 0.11 | 0 | 0.11 |
| X1 | 0.11 | 0.01 | 0.10 |
| X3 | 0.11 | 0.03 | 0.08 |
| X5 | 0.11 | 0.05 | 0.06 |
| X6 | 0.11 | 0.06 | 0.05 |
| X8 | 0.11 | 0.08 | 0.03 |

Following polymerization, the pre-polymer is purified by precipitation in deionized (DI) water, lyophilized, and dissolved in organic solvent to form pre-polymer solutions. The CXBE prepolymer is then dissolved in organic solvent including but not limited to dioxane, acetone, ethanol, and ethyl acetate. Alternatively, CXBE prepolymers may be synthesized for set periods of time (e.g., from fifteen minutes to 1.5 hours) followed by quenching in ice to yield a viscous liquid. The prepolymers are then cast into desired shapes via solvent casting, dip coating, etc. to form solid materials. The solid materials may then be further cross-linked. This can involve thermal cross-linking.

Other embodiments can include biodegradable photoluminescent polymer (BPLP) prepolymers (synthesized as above with addition of the appropriate amino acid and utilized as CXBE) and urethane chain extended CXBEs (synthesized as above with the addition of isocyanate chain extenders/crosslinkers following prepolymer synthesis).

Referring to Table 2, citrate based polyesters may be synthesized via the general procedure(s) discussed above using a variety of diols, polyols, and other functional monomers. Suitable diols can be small molecule diols such as 1,2-ethylene glycol, 1,3-propanedio1,1,4-butanediol, 1,5-pentane diol, 1,6-hexanediol, 1,8-octanediol, 1,10-decanediol and 1,12-dodecanediol or macrodiols such as poly(ethylene glycol) (PEG) or combinations thereof.

TABLE 2
Molar Ratios of Various Citrate Based Polyester Formulations

| | Citric Acid | Xylitol | Octanediol | |
|---|---|---|---|---|
| | (mols) | (mols) | (mols) | PEG |
| X6 | 0.11 | 0.06 | 0.05 | 0.00 |
| P1 | 0.11 | 0.06 | 0.04 | 0.01 |
| P2 | 0.11 | 0.06 | 0.03 | 0.02 |

Polymers may be synthesized with citrate: diol ratios of 1.5:1 to 1:1.5. BPLPs may be synthesized with a variety of amino acids or similar monomers at varying ratios. A variety of isocyanates may be used including hexamethylene diisocyanate, isophorone diisocyanate, or other polyisocyanates. The isocyanate to polymer ratio may be varied. Additional monomers including but not limited to dopamine, L-DOPA, azide, alkyne, aniline oligomer, etc. may be incorporated at varying ratios.

Citrate based polyesters may be synthesized via the general procedure above using a variety of water-soluble diols including poly(ethylene glycol). Prepolymers may be solubilized in solvents including water, acetone, dioxane, ethanol, PEG dimethyl ether, ethyl acetate, etc. at varying concentrations. Unsaturated monomers, amine or thiol-containing monomers, and a variety of other monomers may be partially or wholly substituted in the above synthesis. Substituted monomers may further comprise catalysts such as tertiary amines for urethane synthesis. Substituted monomers including but not limited to PEG and N-methyldiethanolamine (MDEA) may be incorporated to modulate polymer degradation.

Citrate based polyesters may be mixed with a variety of porogens including but not limited to salts, particles such as PVA, PVP, small molecules such as poly(ethylene glycol) dimethyl ether, or combinations thereof to impart porosities of various sizes and shapes to three dimensional constructs.

Citrate based polyesters may be mixed with a variety of agents including but not limited to ceramics, metals, metal oxides, nano- or microparticles, graphene oxides, or carbon nanostructures. Such additives may alter mechanical or other physical properties, may impart functionalities including fluorescence, absorbance, photothermal effects, electrical properties or sensing properties. These or other additives may provide additional benefits through the release of constituent or incorporated products such as ions, drugs, or growth factors.

Polyester constructs may be further polymerized via thermal polymerization, urethane/urea crosslinking, click crosslinking, free radical crosslinking, as well as other methods or a combination of methods to form the final product.

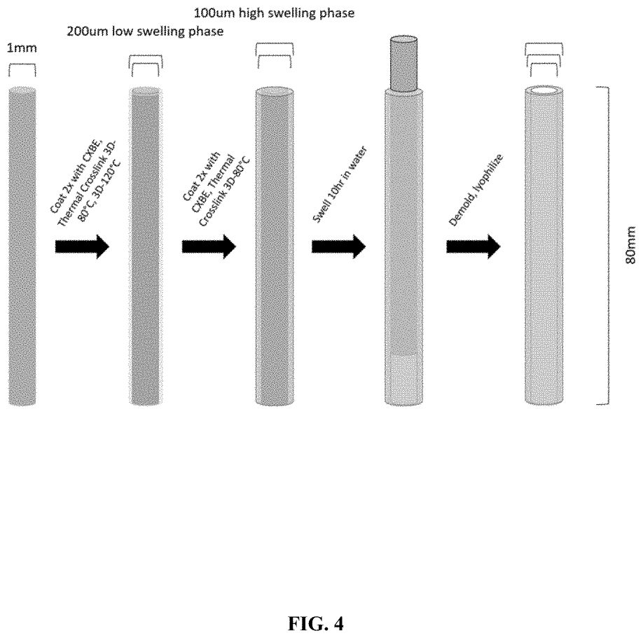

Referring to FIG. 4, in an exemplary embodiment, CXBE prepolymer can be synthesized via a polycondensation reaction of citric acid, xylitol, and octanediol followed by purification to obtain a viscous liquid. With this exemplary embodiment, NaCl, with a particle range of 50 um, is synthesized and mixed with CXBE prepolymer (30:70 CXBE: NaCl wt: wt) to form a viscous slurry. The CXBE catheter 102 is fabricated by first dip-coating rods with CXBE prepolymer to form a solid inner phase (200 um thick) followed by dip coating the rods with a CXBE/NaCl slurry to form an outer porous phase (100 um thick). The CXBE catheters 102 are then polymerized followed by demolding and porogen leaching.

Referring back to FIG. 2, it can be beneficial to measure the effects and functions of the catheter 102 and/or the PNB device 100. Furthermore, most surgical procedures are invasive and do not provide a means for monitoring and evaluation over time. Thus, it may be desired to have a PNB device 100 with the capability for monitoring and providing feedback. It may also be desired to have a PNB device 100 that is capable of providing precisely controlled applications of stimuli in diverse locations. Embodiments of the PNB device 100 disclosed herein provide a material platform capable of prolonged support and access points for monitoring, treatment delivery, and treatment modulation while evading surgical removal or permanent implantation.

Embodiments of the catheter 102 can include monitoring systems 110, delivery systems 112, and/or modulation systems 114. Such systems are contemplated to be biodegradable, but they need not be. Any one or combination of these systems can be configured to operate autonomously (e.g., include shape memory materials as switches that actuate the system upon a condition being set) or via controlled operation of an operating module 116. The operating module 116 can be in wireless communication with a wireless module 118 (e.g., biodegradable transceiver) embedded within or disposed in the catheter 102. Any of the monitoring systems 110, delivery system 112, and/or modulation systems 114 can be in electrical connection with the wireless module 118 via biodegradable electrically conductive moieties (e.g., aniline oligomers or polymers, carbon nanostructures, silver nanostructures, graphene oxides, and copper nanostructures, other metallic films, etc.). Thus, signals can be transmitted to and from the operating module 116 and the systems 110, 112, 114 via the wireless module 118.

Some embodiments include incorporating moieties within the CXBE material of the catheter 102 to serve as a monitoring system 110. These moieties can be in the form of sensors. Thus, the monitoring system 110 can include sensors embedded within the CXBE material of the catheter 102. In addition, or in the alternative, the monitoring system 110 can include separate sensors delivered via the PNB device 100. This can involve delivery of the sensors via the lumen 104. It is contemplated for the sensors to be biodegradable, but they need not be. The biodegradable sensors can be zinc (Zn)-based, magnesium (Mg)-based, tungsten (W)-based, molybdenum (Mo)-based, iron (Fe)-based, etc. The sensing can measure pressure, temperature, electrical impulses, infections, pH, blood oxygen levels, chemicals, ions, etc.

For instance, embodiments of the catheter 102 can have traces or films of biodegradable metallic material embedded within the CXBE material. The traces can have predetermined temperature coefficients of resistance. The arrangement of traces can be used to generate a temperature sensor array. As fluid flow leads to anisotropic thermal transport phenomena, the flow of blood or other body fluids can be accurately quantified using the temperature sensor array. The resulting temperature measurements in the range from 25 to 50° C. can be associated with a high precision of 0.01° C., for example. Some embodiments can include a thermal generator in electro-mechanical connection with the temperature sensor. The temperature sensor can serve as a thermal actuator to allow the thermal generator to provide a mild, well-controlled modulation of temperature of the tissue 106 within which the catheter 102 is inserted. In addition, responses of the other temperature sensors can be used to determine spatiotemporal distributions of temperature that result from anisotropic thermal transport heating. For instance, two temperature sensors can be positioned along the flow direction to provide for differentiated temperature readings.

As another example, embodiments of the catheter 102 can have traces or films of biodegradable metallic material embedded within the CXBE material, the traces can be configured to operate as electrodes (e.g., a working electrode and a reference electrode). A potential difference between the working and reference electrodes can generate a signal that is representative of changes in pH. The electrodes can be configured to operate as a potentiometric pH meter—electric current generated by the electrodes can be dependent upon hydrogen-ion activity, indicating acidity or alkalinity expressed as pH. An ion-selective (or permselective) membrane on the working electrode can enable measurement of concentrations of corresponding cation/anion (or hydrogen evolution), for example.

As another example, embodiments of the catheter 102 can have traces or films of biodegradable metallic material embedded within the CXBE material, the traces configured to operate as electrodes for measurement or monitoring of electrical impulses. For instance, some PNB devices 100 can include the use of electrical impulse therapy or treatment—electrical stimulation for nonpharmacological neuroregenerative therapy. Direct intraoperative electrical stimulation of injured nerve tissue 106 proximal to the site of repair has been demonstrated to enhance and accelerate functional recovery. Such a sensor can monitor electrical impulses being delivered. In addition, or in the alternative, the electrodes can be used to generate and deliver the electrical impulses to the surrounding tissue 106. The generation of the electrical impulses can be controlled via the operating module 116 for proper nerve stimulation. Such smart PNB devices 100 can substantially improve patient care by quantitative assessing and enhancing pain control in a closed-loop manner. This can greatly enhance neural or other tissue 106 stimulation, as well as the guidance of the surgical placement.

As another example, embodiments of the catheter 102 can have biodegradable light emitting diodes (LEDs) and biodegradable circuitry to form a photodetector that generates a signal based on blood oxygenation concentration. For instance, the sensor can measure blood oxygenation optically via the Lambert-Beer law and generate a signal representative of the blood oxygen concentration.

Some embodiments can include encapsulated sensors. For instance, any one or combination of the sensors discussed herein can be encapsulated with a biodegradable encapsulation layer (e.g., poly(lactic-co-glycolic acid) (PLGA)). This can be done to provide a two-stage degradation. The encapsulation insolates the sensor for proper and stable operation until the encapsulation layer is degraded. The full degradation of the sensor and catheter 102 can then follow.

Some embodiments of the catheter 102 can include a delivery system 112. The delivery system 112 can be shape memory material embedded within the catheter 102 at or near the lumen 104. The shape memory material can be configured to encapsulate (e.g., molecular encapsulation) an agent and controllably release the agent (via a change in molecular structure for example) upon the material being exposed to a condition (e.g., a temperature, a pressure, a pH level, a blood oxygen level, change in electric field, change in magnetic field, etc.). Once the environment changes to remove the material from the condition, the shape memory material resumes its state in which no release of the agent occurs. As another example, the delivery system can be a matrix of the disclosed biodegradable crosslinked polymer within the catheter 102. The biodegradation rate of the matrix can be controlled or tunable, thereby allowing for tunable and continuous release of an agent encapsulated therein to the surrounding tissue 106 area.

The agents can be drugs and other chemicals (analgesics, antimicrobial agents, chemotherapeutics, and pharmaceuticals, anti-inflammatory agents, hydrogels, nanoparticles, etc.), growth factors (naturally occurring substances capable of stimulating cell proliferation, wound healing, and occasionally cellular differentiation), cells, bioactives, etc. The agents can be introduced via direct mixing, chemical conjugation, incorporated within particles, etc.

In some embodiments, the catheter 102 can be embedded with agents that are released via diffusion. These agents can include antimicrobial polymers, agents, etc. to reduce or eliminate implant-associated bacterial infection, provide anticancer treatments, etc. For instance, CXBEs can be combined with the anesthetic Lidocaine via physical mixing of the prepolymer solution and Lidocaine powder dissolved in ethanol to form a homogeneous mixture that can be further utilized for fabrication of the catheter 102. Alternatively, Lidocaine may be directly reacted during polycondensation, yielding a homogeneous prepolymer.

Bioactives can be an agent for delivery via the delivery system 112. The bioactive can be introduced for direct and local modification of cells and tissues 106. Such use can be used for biology and tissue 106 engineering. For instance, direct injection of deoxyribonucleic acid (DNA) and ribonucleic acid (RNA), either free or contained, within a biodegradable matrix can have myriad and important effects in basic research, as well as regenerative medicine. Introduction of DNA or RNA within a biodegradable material, as though an embodiment of the catheter 102, can alleviate the need for invasive surgeries.

In an exemplary embodiment, a bioactive (e.g., DNA or RNA) can be encapsulated in a biodegradable matrix within the catheter 102. The matrix can biodegrade and release the bioactive via the lumen 104. The DNA or RNA can then be introduced into a relatively non-mobile tissue 106 or organ (including skeletal muscle, salivary glands, the spinous process surrounding the dorsal root ganglion neurons, subcutaneous tissue, tumors and the small or large intestine, etc.). The degradation rate of the matrix can be controlled or tunable, thereby allowing for tunable and continuous release of the encapsulated agent into the surrounding tissue 106 area. The bioactive can then be uptaken into cytoplasm and nuclei of the tissue's cells. Delivered DNA could acutely transfect the organ/tissue of interest to examine the effect of gene functions, while RNA could knock out proteins of interest in an acute manner. The patient could thus be used as an ‘incubator’ rather than transfecting ex vivo. Optimization of this method of gene transfer can reduce or eliminate the use of potentially toxic or lethal viruses or viral vectors. Additionally, such methods can minimize unpredictability and maximize efficacy as well as potential immune reactions.

Using embodiments of the biodegradable catheter 102 can provide for sustained delivery of such bioactives—i.e., bioactives can be delivered over an extended period of time, allowing greater control as well as enhancing the efficacy of treatment versus single injections (which often fail to provide adequate delivery or result in the loss of a large amount of the delivered cells, DNA, etc. due to migration or destruction). In addition, such delivery techniques can provide for ready delivery of multiple doses of bioactives as necessary to counteract such limitations. In addition, any one or combination of the sensors disclosed herein can further allow for dynamic monitoring throughout the course of treatment.

Additional bioactives can include cells, proteins, growth factors, etc. Any one or combination of the bioactives can be loaded into a carrier (e.g., nanoparticles) to provide sustained release of the bioactive after the bioactive has been delivered to the tissue.

Some embodiments of the catheter 102 can include a modulation system 114. The modulation system 114 can be any factor that controls the release of an agent, controls the effects of an agent, counteracts the effect of a condition, etc. The activation of the modulation system 114 can be based on a condition experienced by the modulation system 114 (e.g., the modulation system can be shape memory material), based on sensor signals from the monitoring system 110 that are processed by the operating module 116, and/or based on manual inputs from a user of the operating module 116 (e.g., the operating module 116 can be a computer device with a user interface) that are transmitted as command signals via the wireless module 118.

In an exemplary embodiment, the modulation system 114 can be a shape memory material embedded within the catheter 102 and disposed at a periphery of the lumen 104. The shape memory material can be activated to constrict the lumen 104 or activated again to dilate the lumen 104. The constriction or dilation can be used to control the flow of fluid through the lumen 104.

As another example, biodegradable circuitry can used to create electrical, magnetic, photothermal, photoacoustic, light, etc. stimuli. These stimuli can be used to dynamically modulate agent release, material properties of material used to fabricate the catheter 102, material properties of encapsulations, material properties of tissues/cells, etc. For instance, the catheter 102 can include materials having dielectric/piezoelectric properties, being capable of modulation by polymer structure, additives, or polarizable bonds such as ureas. Said properties can be modulated by mechanical loading (flexoelectricity), such that, under physiological loading conditions, electrical potentials can be used to influence cells toward locomotion, differentiation, etc. These stimuli can also be used to elicit certain responses from tissues/cells, modify absorption rates, modify diffusion rates, modify degradation rates, etc. In some embodiments, the stimuli can be used to generate therapeutic responses in tissue 106.

As another example, the catheter 102 can incorporate chemical structures responsive to light, temperature, pressure, electricity, pH, etc. Such responses can be used as stimuli to control release of agents, to effect changes in the catheter's structure or function (e.g., reversible networks/crosslinks, dynamic changes in mechanics or degradation rate, activation of self-healing or shape memory properties, etc.), etc.

As another example, the catheter 102 can incorporate ceramics, metals, metal oxides, etc. capable of generating oxygen, hydrogen, bioactive ions, etc. to affect cells and tissues 106.

In some embodiments, the lumen 104 are structured to incorporate ridges, valleys, surface roughness, or other three-dimensional structures onto the interior lumen 104 surface. This can be done to modulate liquid flow, which can include modulation via capillary action. As another example, shape memory material can be formed on an interior surface of the lumen 104 so that when activated ridges, valleys, surface roughness, or other three-dimensional structures are generated but the interior surface is otherwise smooth.

Some embodiments of the catheter 102 can include biodegradable circuitry in the form of a power harvester 120. This can be a radio frequency power harvester 120, for example. The power harvester 120 can be used to provide electrical power to operate any one or combination of the monitoring system 110, delivery system 112, modulation system 114, or wireless module 118. In some embodiments, the wireless module 118 includes the power harvester 120. In an exemplary embodiment, the power harvester 120 includes an inductor (Mg or other biodegradable metallic coils), a radio frequency diode (Si nanomembrane active layer; biodegradable metal electrodes), a capacitor (SiO2 dielectric layer and biodegradable metal layers), and a biodegradable substrate (such as PLGA) interconnected with biodegradable metal traces. The power harvester 120 can be in electrical connection with any one or combination of the monitoring system 110, delivery system 112, modulation system 114, or wireless module 118 via metallic traces. While the power harvester 120 can be part of the catheter 102, it can also be inserted into the tissue 106 or other portion of the patent and be placed into electrical connection with components of the catheter 102 via percutaneous wiring. The percutaneous wiring can be biodegradable molybdenum wires 10 micrometers thick or magnesium wires 50, for example.

PNB devices 100 and associated catheters 102 made with the CXBE, along with the monitoring system 110, delivery system 112, and modulation system 114 provide for an improved continuous PNB device 100 that reduces complications and broadens clinical use, reducing hospitalization duration and associated costs, reducing use and potential abuse of opioids, and improving patient pain management and postoperative recovery through the following: (1) use of a biomaterial exhibiting high strength and rapid degradation via polymer network engineering; (2) use of a biodegradable biphasic PNB catheter 102 via a fabrication method that will be discussed later; (3) use of a smart PNB catheter 102 with integrated sensors and stimulation modules with closed-loop control; and/or (4) the development of a clinically relevant PNB devices 100 for improved continuous neural block for surgical procedures and drug delivery applications such as temporally and spatially controlled delivery of analgesics and chemotherapeutics, as well as sensing applications including monitoring of postanastamosis hypoxia.

In addition to material swelling (discussed supra), adherence to and/or anchorage within the local tissue can be achieved via other anchoring mechanisms. These can include physical and/or chemical adherence techniques. For instance, the outer surface of the catheter 102 can be structured with surface roughness or include nano- or micro-structures such as hooks or grooves. Some embodiments of the catheter outer surface can include porous structures, allowing tissue infiltration. Some embodiments of the catheter outer surface can include adhesive moieties configured to promote cell or tissue binding. These moieties can include shape memory material that form hooks, loops, barbs, etc. when activated.

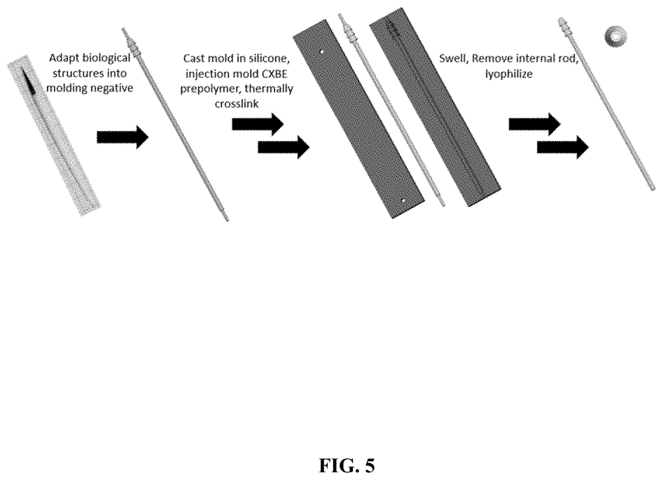

FIG. 5 shows an exemplary fabrication procedure for generating a CXBE catheter 102 with a biomimetic anchor design. The CXBE catheter 102 can be fabricated with biomimetic anchor designs including but not limited to porcupine quills, honey bee stings, etc. Such catheters 102 can be fabricated via a negative molding of the desired structure in silicone followed by injection molding of CXBE prepolymer, thermal crosslinking, and demolding to achieve the final desired hollow catheter.

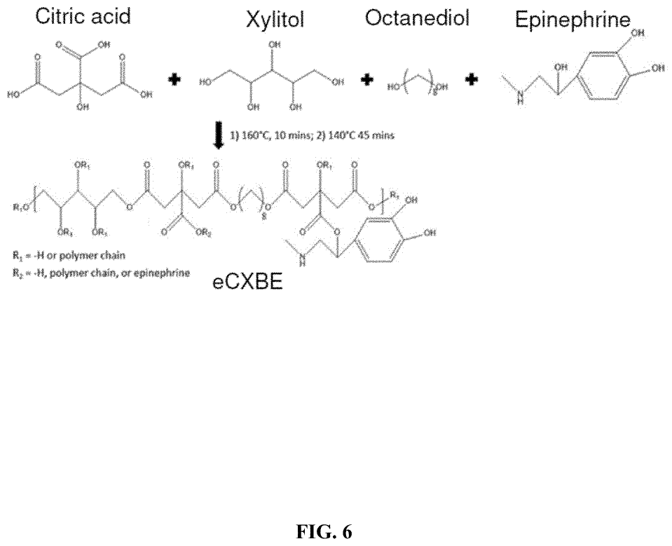

An exemplary tissue 106 adhesion moiety can be the incorporation of epinephrine. FIG. 6 shows a fabrication method that incorporates epinephrine into CXBEs through an esterification reaction to generate epinephrine bearing (eCXBE) polymers. Bupivacaine can be encapsulated within eCBXE (eCBXE/bupivacaine) to provide sustained release and facilitate a post-operative nerve block. Bupivacaine (0.065% wt/vol (650 ug/mL)) is commonly used in PNB devices 100 in combination with epinephrine (5 ug/mL) due to the latter's vasoconstrictive effects, which serve to decrease bupivacaine clearance and consequently enhance its effectiveness, thus rendering the two drugs ideal candidates for incorporation into CXBEs. Epinephrine further contains both hydroxyl and catechol functional groups, the former enabling ready reaction with pendent carboxyl groups of citrate and the latter providing pendant catechol functionality enabling tissue adhesion similar to canonical mussel inspired adhesives. Epinephrine is thus capable of playing a dual role in catheter 102 design, supplementing swelling based anchorage while enhancing bupivacaine mediated nerve block functions.

eCXBE prepolymer can be synthesized via polycondensation reaction of citric acid, epinephrine (0-0.03mol ratio to citrate), xylitol, and octanediol followed by purification to obtain a viscous liquid. Bupivacaine can be mixed with eCXBE at various wt % (10-30) to obtain eCBXE/bupivacaine. eCXBE/bupivacaine catheters can be fabricated by first dip coating rods with CXBE prepolymer to form the 300 um thick tube. In order to localize bupivacaine release to the nerve, the proximal half of the catheter 102 can be fabricated with eCBXE/bupivacaine while the distal half can be coated with eCBXE sans bupivacaine. eCXBE/bupivacaine catheters can then be polymerized followed by demolding. SEM imaging of fabricated catheters can be used to determine successful fabrication of the tubular structure. Swelling ratio can be determined via immersion of samples in phosphate-buffered saline (PBS) at 37° C. with measurements at 1, 3, 7, 10, 14, 18, and 21 days followed by weekly measurements until equilibrium is reached (3 consecutive time points with no change). Degradation can be measured by incubation in PBS (with weekly PBS change) at 37° C., with mass loss assessed for weekly 4 weeks. pH evolution can be determined by measuring the pH of the PBS at 1, 3, 5, and 7 days followed by weekly measurements. Release of bupivacaine and epinephrine can be determined by incubation in PBS at 37° C. with release measured at 1, 3, 6, 9, and 12 hours and 1, 3, 5, 7, 10, 14, 18 and 21 days via high performance liquid chromatography (HPLC). Tensile mechanical properties can be measured using an Instron mechanical tester in both dry and equilibrium swollen conditions. Adhesion strength can be determined using a modified lap shear method.

Fabricated CXBE catheters 102 display homogeneous structures. Degradation rate and swelling of CXBE-bupivacaine catheters can be modulated through adjustment of monomer ratios and crosslinking time (1d 80° C. to 3d 80° C.+3d 120° C. under vacuum). Mechanics may also be tuned in the above manner. Lap shear adhesion strength can be increased by increasing epinephrine content or conjugating epinephrine directly to the catheter surface using 1-Ethyl-3-(3-dimethylaminopropyl)carbodiimide (EDC) chemistry to increase catechol functional groups. Bupivacaine and epinephrine release may be modulated by adjusting drug loading, catheter crosslinking time, or by encapsulating bupivacaine within nanoparticles prior to loading to prolong release.

Similarly, lidocaine can be encapsulated within eCBXE (eCBXE/lidocaine) to provide sustained release and facilitate a post-operative nerve block.

Some embodiments of the catheters 102 to be composed of multiple distinct layers or phases, including solid and porous, micro or nanostructured surfaces, and layers/phases containing gradient mechanical and/or degradation rates. For instance, distal ends of the catheter 102 may be layered to exhibit a mechanical strength and/or degradation rate that differs from that of the proximal end of the catheter 102. As another example, the catheter 102 may be layered so that a side of the catheter 102 exhibits a mechanical strength that differs from another side so as to bias the catheter's 102 deflection in a predetermined direction.

It is contemplated for embodiments of the catheter 102 to be imaged through Magnetic resonance imaging (MRI), ultrasound imaging, fluorescence imaging, photoacoustic imaging, etc.

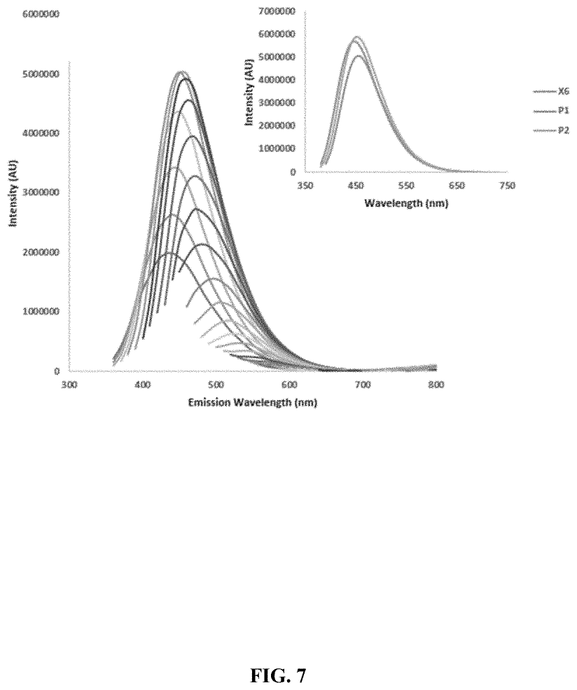

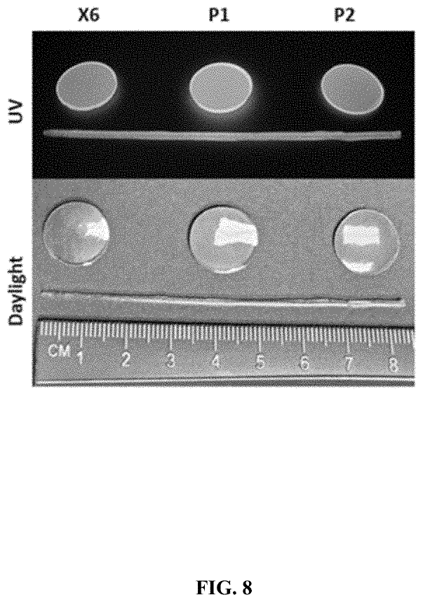

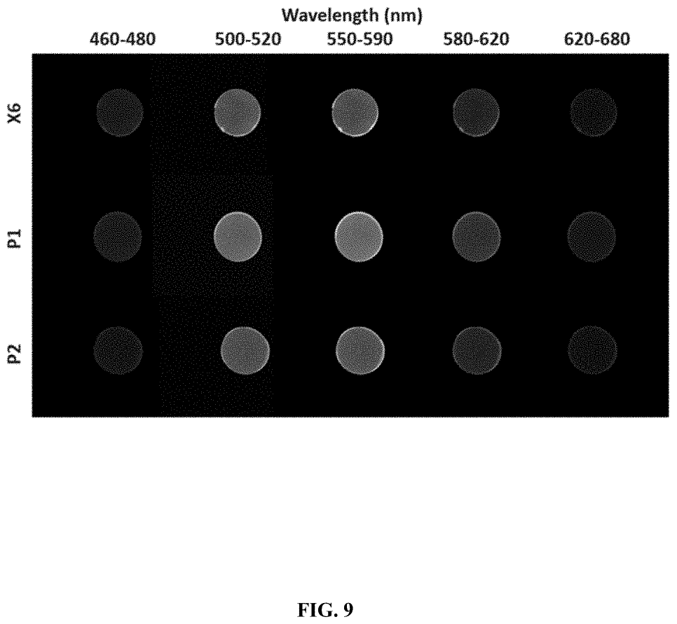

For instance, some embodiments of the catheter 102 can incorporate fluorescence via polymer moieties or additives. This can allow the catheter 102 to be more easily viewed for positional and degradation tracking. This can also allow the catheter 102 to provide a source of light. CXBEs may be synthesized to obtain highly fluorescent polymers displaying emissions ranging from 350 nm to 650 nm (see FIG. 6) and bright fluorescence under ultraviolet illumination (see FIG. 7). CXBEs display band shifting behavior, allowing them to fluoresce at a wide variety of wavelengths from blue to red (see FIG. 8). This intrinsic fluorescence enables multiple functions including in vivo imaging as well as direct light delivery to the local nerve via illumination of the catheter 102. Such light delivery can also be used in antimicrobial applications as well as optogenetic stimulation of nerves.

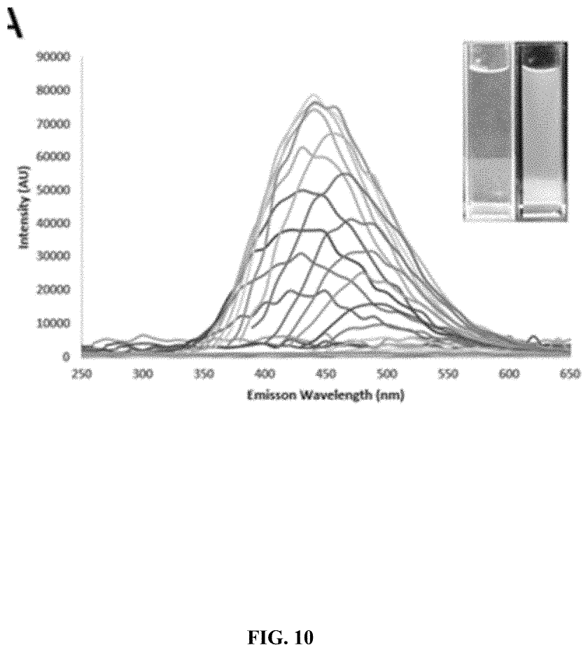

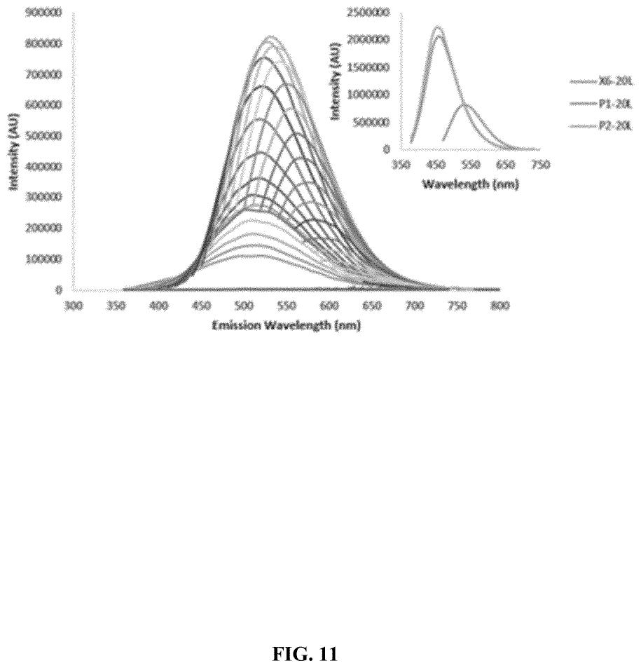

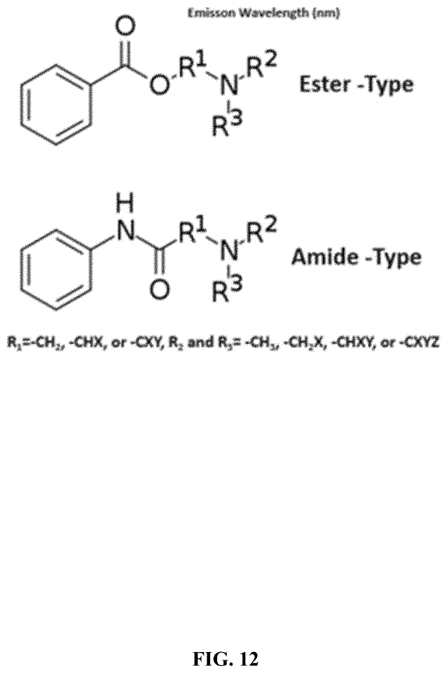

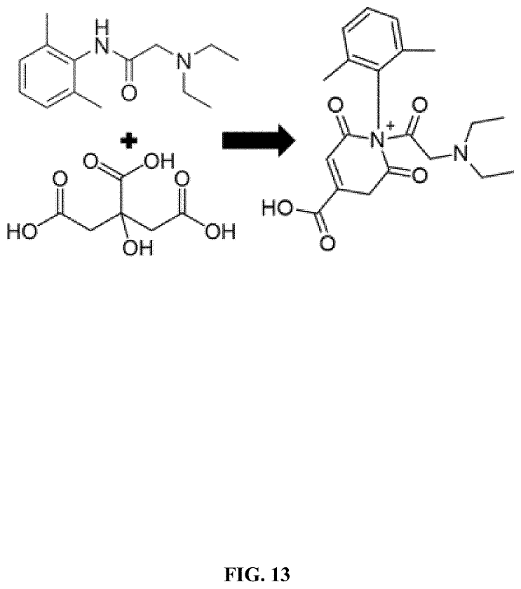

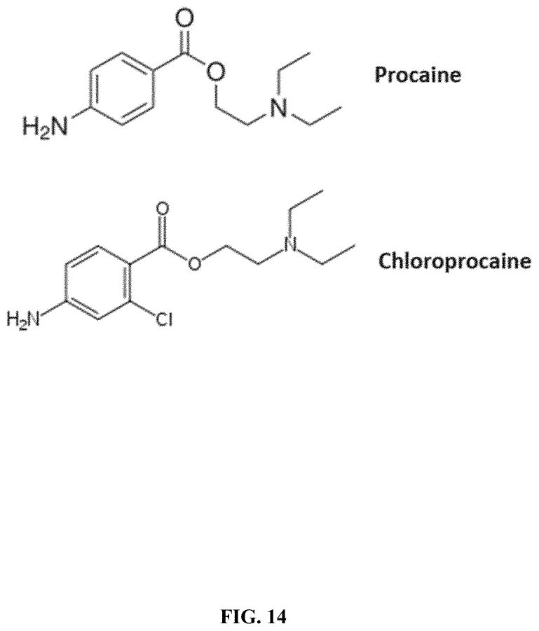

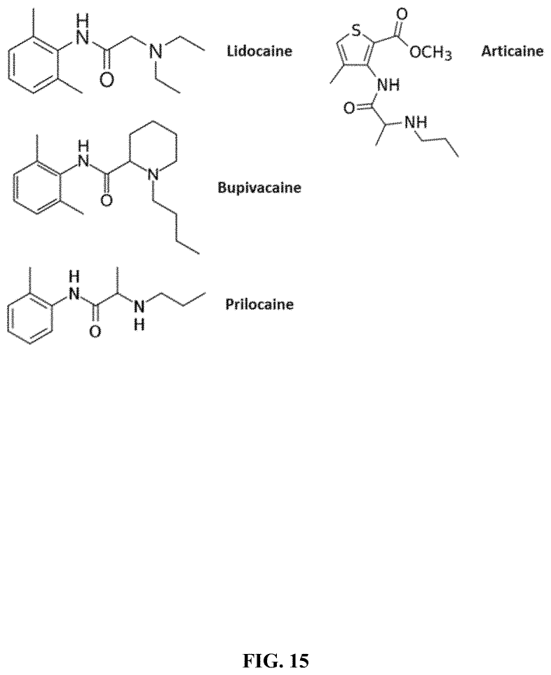

In some embodiments, CXBEs can be combined with various anesthetics to synthesize fluorescent prepolymers, polymers, and catheters 102 capable of both drug release as well as intrinsic imaging. Representatively, the direct polycondensation reaction of citric acid and lidocaine results in a highly fluorescent, band shifting molecule with emissions ranging from 250 nm to 650 nm (see FIG. 10). Additionally, the crosslinking of CXBEs physically mixed with lidocaine as described herein yields a fluorescent structure with strong emission from 450 nm to 650 nm (see FIG. 11). Given the typical primary and secondary amine containing structures of anesthetics of both the ester- and amide-type (see FIG. 12), it is the reaction of citric acid with a multitude of anesthetics via said amines that results in such fluorescent materials and molecules. FIG. 13 shows a structure for CA-Lidocaine and FIGS. 14 and 15 display representative anesthetics of the ester- and amide-type, respectively, capable of potential reaction with citric acid toward the synthesis of fluorescent molecules and materials for imaging.

In some embodiments, the catheter 102 incorporates moieties or additives capable of absorbance in the infrared region including but not limited to aniline carbon nanostructures, serine, graphene oxide, copper, silver, gold, cobalt, and iron in order to enable photoacoustic imaging as well as photothermal effects.

Some embodiments of the catheter 102 incorporate moieties or additives including but not limited to iron oxide capable of magnetic response to generate local magnetic fields and photothermal effects.

Some embodiments of the catheter 102 incorporate moieties or additives that provide antibacterial and/or antifungal properties.

### EXAMPLES

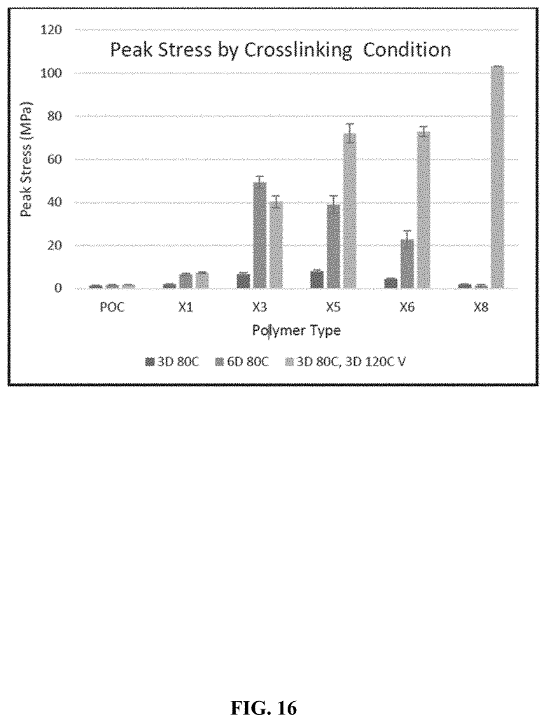

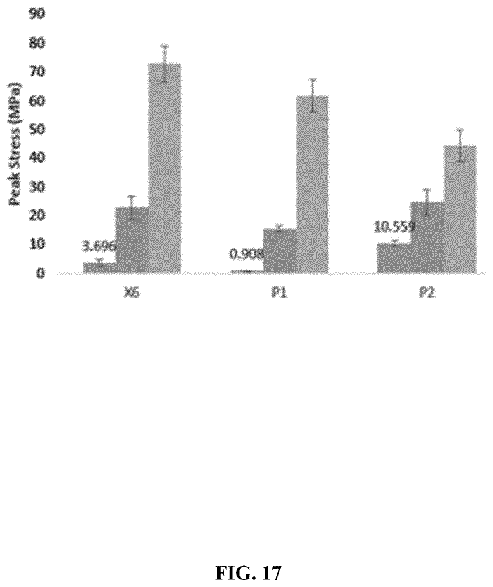

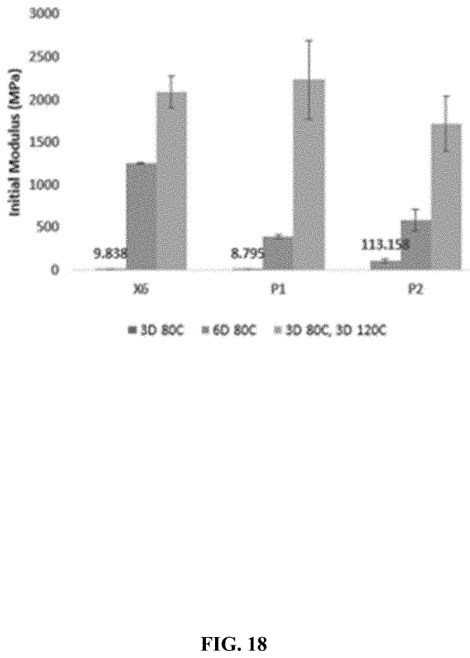

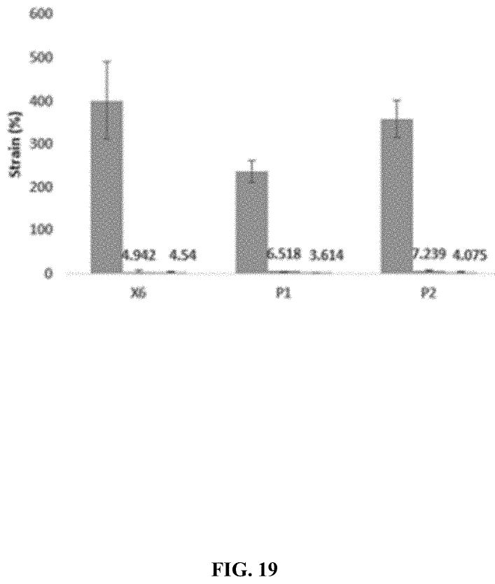

CXBEs were successfully synthesized via a simple, economical, and environmentally friendly polycondensation reaction of citric acid, xylitol, and octanediol (1:1 mol ratio of citrate:(octanediol+xylitol). The obtained CXBE prepolymers were subsequently thermally crosslinked to fabricate homogeneous films for testing. Varying the molar feeding ratio of xylitol from 0 to 0.8 mols resulted in an increase in film tensile mechanics from 2.5 MPa to 100 MPa (superior to commercially available biodegradable poly(lactides), previous CBEs, and a commercially available polyurethane PNB catheter (Pajunk 211285-40E, 48 MPa)) (see FIGS. 16-17). Additionally, it may be noted that altering the polymer feeding ratios or crosslinking time results in a wide range of initial tensile moduli ranging from kPa to GPa and tensile strains ranging from over 300% to <5% (see FIGS. 18-19), demonstrating the wide degree of tunability of CXBEs to various tissue environments ranging from soft tissue (nerve, skin) to hard tissues 106 (bone). An additional benefit of this tunability arises when considering sensor integration. Particularly for piezoelectric or other mechanics based sensors, it is critical that the modulus of the material upon which the sensor is embedded or encapsulated matches closely that of the tissue or structure being monitored to prevent stress shielding based mismatches in measured and actual forces.

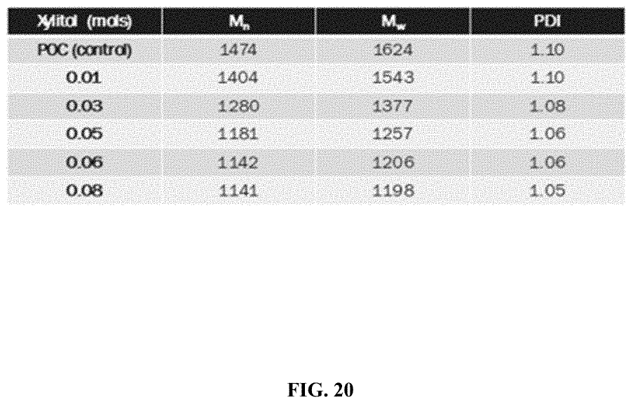

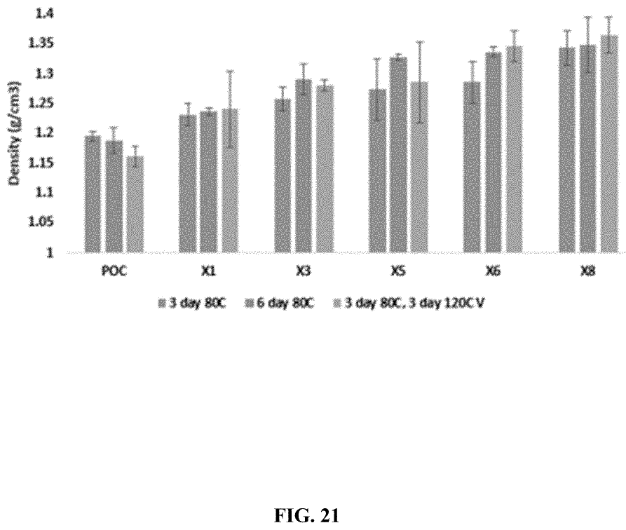

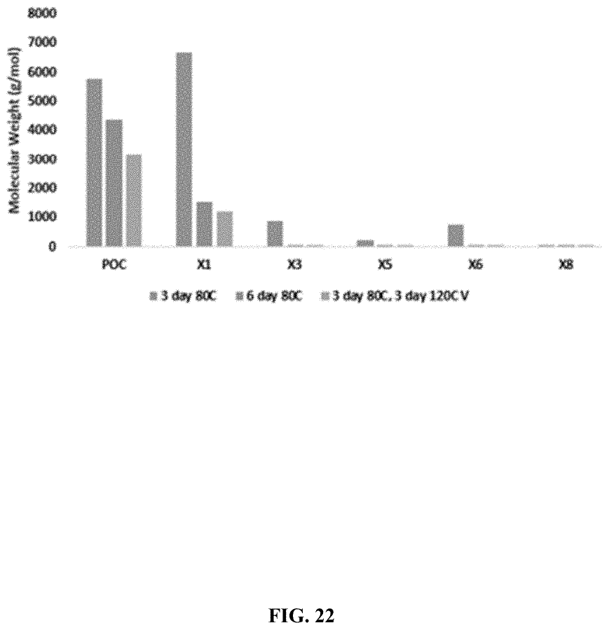

Mass spectroscopy and density measurements of CXBE prepolymers and films, respectively, revealed decreased molecular weight and increased density with xylitol incorporation while the determination of the molecular weight between crosslinks via application of the theory of rubber elasticity confirmed the engineering of a dense, highly crosslinked elastomer through xylitol incorporation (see FIGS. 20-22). Contact angle and swelling measurements demonstrated increased hydrophilicity and concomitant increased swelling of CXBEs vs POC. Additionally, preliminary studies of CXBE/ceramic composites demonstrated an approximately 10× increase in biodegradation of CXBE composites versus POC composites. This demonstrates that CXBEs having a high strength and a rapid biodegradable rate can be made through facile tuning of polymer structure via incorporation of xylitol into a citrate-based material.

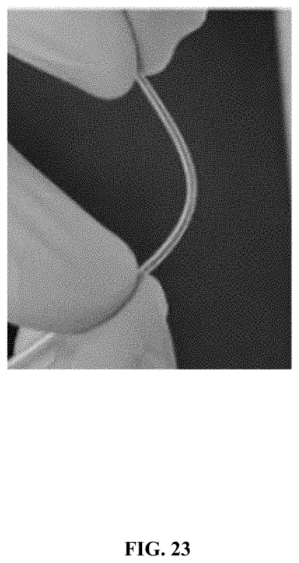

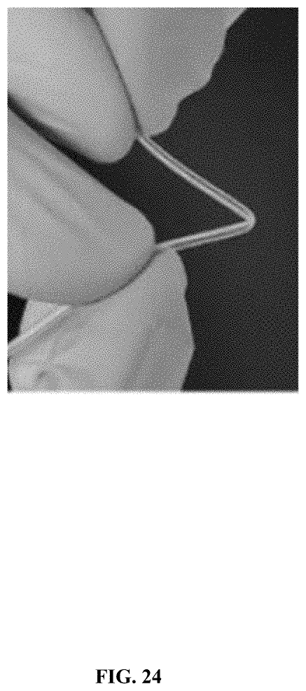

CXBE catheters 102 were fabricated via a simple dip-coating procedure (see FIG. 4). A 1 mm metal rod (80 mm length) was immersed in viscous liquid CXBE prepolymer heated to 80° C. and steadily withdrawn through a 2 mm annulus to create a uniform coating. Subsequently, the coated rod was heated at 60° C. for 12 hours to crosslink the polymer coating and create adherence to the metal tube. Dip coating was repeated three more times followed by thermal crosslinking at 80° C. for three days and 120° C. for one day under vacuum to obtain a catheter 300 micrometers thick. Catheters 102 were removed from the metal rod via swelling in deionized water for 10 hours and were lyophilized to obtain the final CBXE catheter 102. Obtained catheters 102 were readily and repeatedly threaded with a commercial 22-gauge PNB needle and demonstrate significant flexibility and kink resistance compared to commercial polyurethane catheters (see FIGS. 23-24), demonstrating the viability of CXBEs as PNB catheter materials.

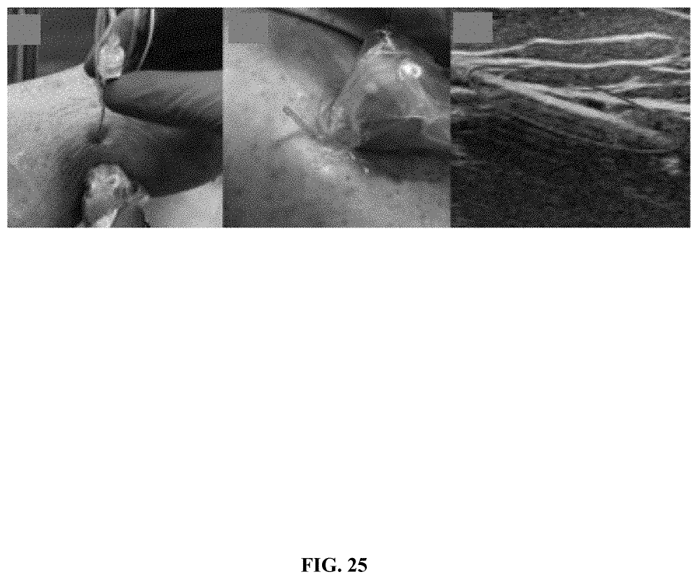

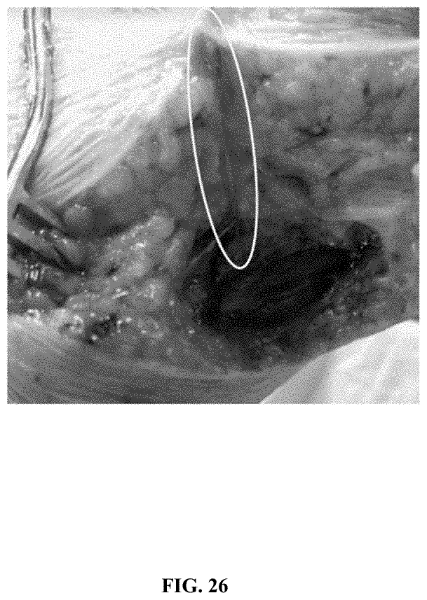

Fabricated CXBE catheters 102 were assembled with a commercially available PNB insertion kit followed by ultrasound-guided insertion into the femoral perineural space of human cadavers to simulate femoral neural block (see FIG. 25). CXBE catheters 102 were readily imaged via ultrasound and thus capable of complete and accurate insertion. Successful withdrawal of the insertion needle sans catheter 102 dislocation or collapse was then achieved. Dissection of the tissue surrounding the catheter 102 revealed it to be intact (see FIG. 26), supporting CXBEs as viable degradable alternatives to current clinical standards.

Test results indicate that catheters 102 made from CXBE can be used for insertion into specific tissue locations including but not limited to nerves, blood vessels, airways, organs such as the heart, lungs, liver, or bladder, subcutaneous tissues, bones, tumors, etc. while maintaining mechanical integrity and interior lumen 104.

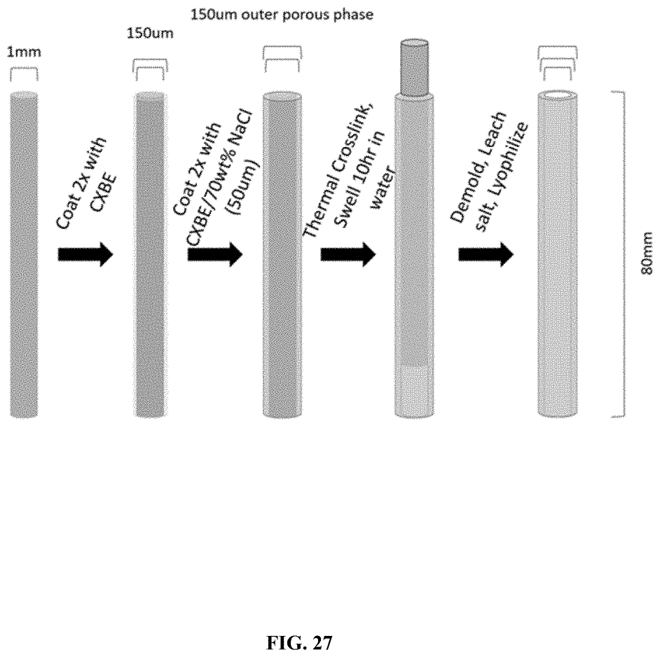

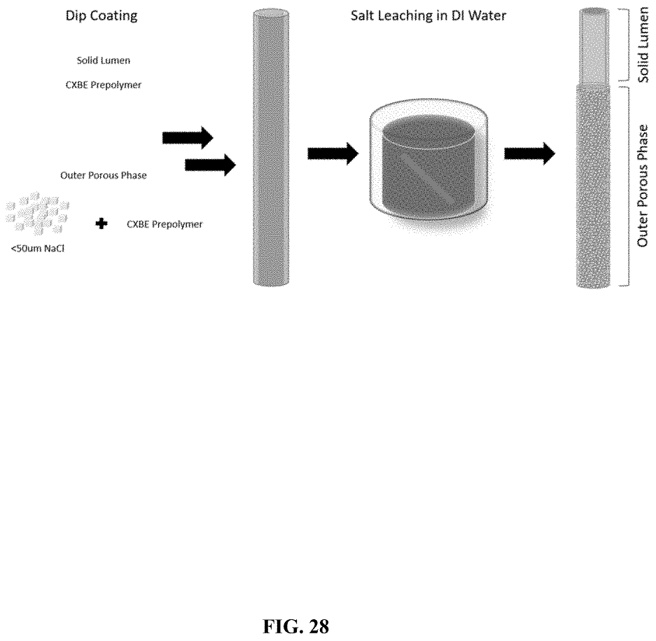

Another method of fabrication can involve synthesizing CXBE prepolymers via polycondensation reaction of citric acid, xylitol, and octanediol followed by purification to obtain a viscous liquid. NaCl with a particle range of 50 um can be synthesized as previously described and mixed with CXBE prepolymer (30:70 CXBE: NaCl wt: wt) to form a viscous slurry. CXBE-b catheters 102 can be fabricated by first dip-coating rods with CXBE prepolymer as described above to form the solid inner phase (150 um thick) followed by dip coating rods with CXBE/NaCl slurry to form the outer porous phase (150 um thick) (see FIG. 27). CXBE-b catheters 102 can then be polymerized as above followed by demolding and porogen leaching (see FIG. 28). Such catheters 102 can provide tissue 106 anchorage via infiltration of the porous outer phase by surrounding cells. It is contemplated for catheters 102 to be fabricated with a variety of porosities, pore sizes, and various porous and non-porous phases.

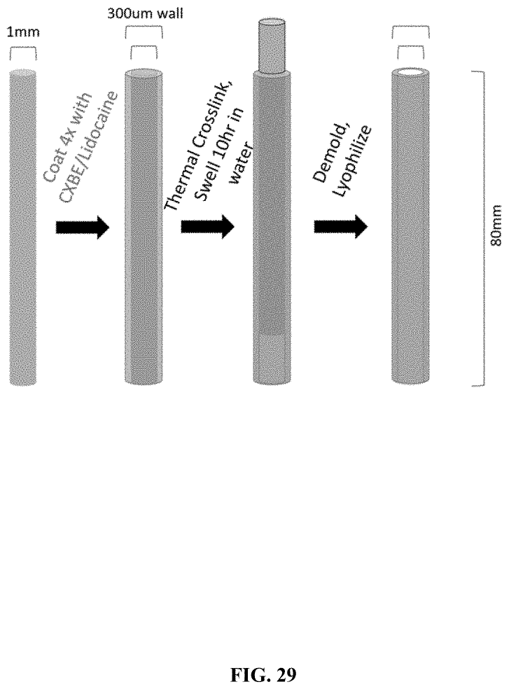

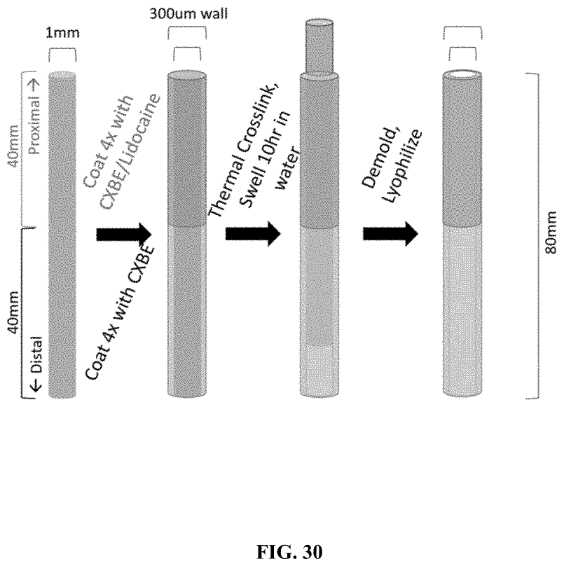

Anesthetic containing CXBE catheters 102 can be fabricated via dip coating of drug containing prepolymers to create single drug releasing phase catheters 102 (see FIG. 29). In addition, or in the alternative, anesthetic containing CXBE catheters 102 can be fabricated via dip coating of drug containing and non-drug containing prepolymers to create catheters 102 containing drug in the location proximal to the nerve but not in the location distal to the nerve, localizing drug release to the immediate nerve adjacent area and reducing likelihood of off-target effects (see FIG. 30).

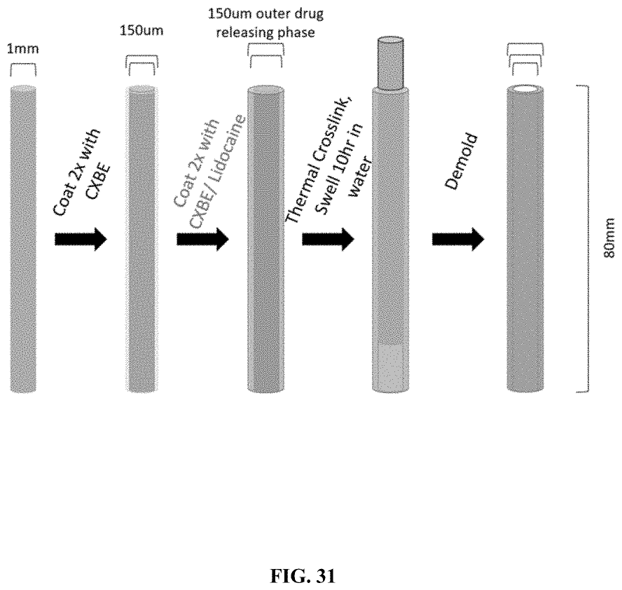

Anesthetic containing CXBE catheters 102 can be fabricated via dip coating of drug containing and non-drug containing prepolymers to create catheters 102 containing drug in the outer phase with the inner phase drug free (see FIG. 31). It is contemplated for the catheters 102 to be fabricated with multiple drug and non-drug containing phases containing multiple drugs as well as incorporating variable phases with respect to swelling, porosity, etc. as described above.

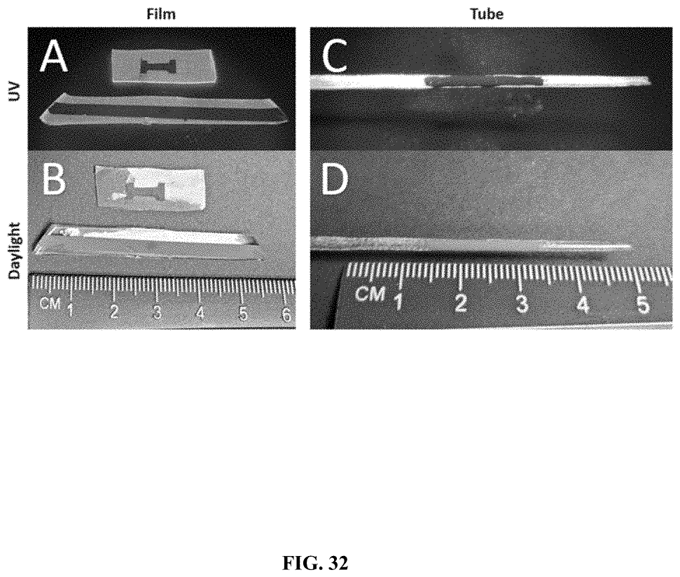

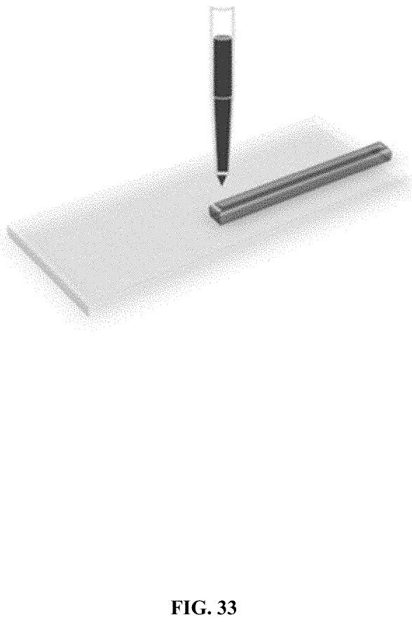

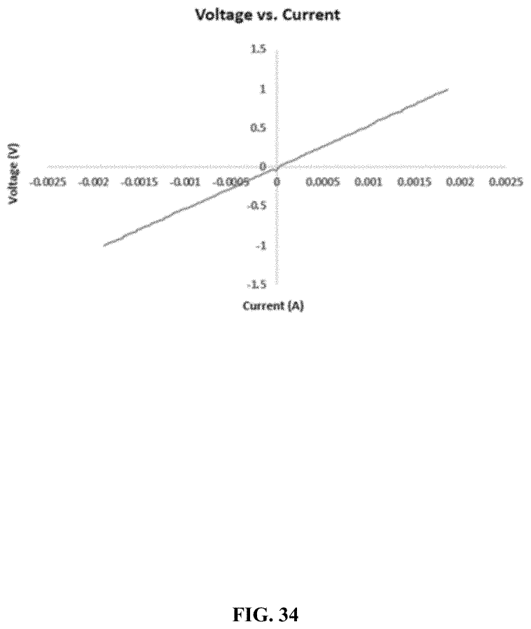

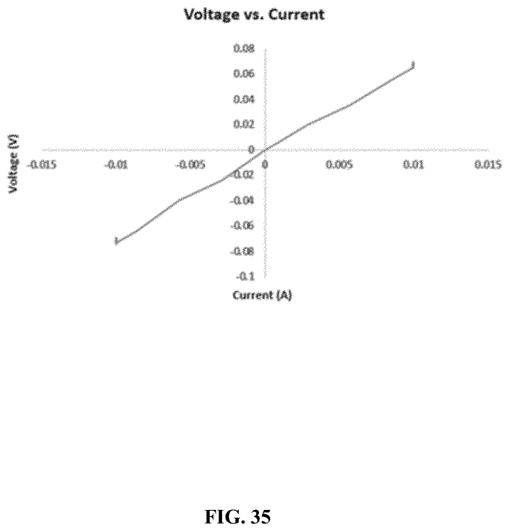

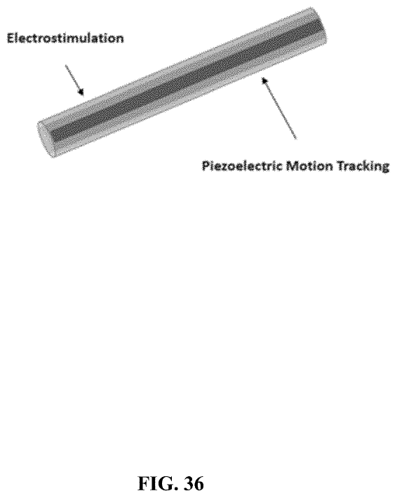

CXBE polymer films and catheters 102 can be readily combined with Zn conductive sensors via a simple liquid deposition technique (see FIGS. 32 and 33). Such techniques may be combined with the use of masks or 3D printing to generate complex geometries. Zn sensors deposited on both CXBE films and catheters 102 display impressive conductivity with negligible resistance (see FIGS. 34 and 35). The impressive conductivity of CXBE/sensor assemblies may be utilized for multiple functions including as electrical stimulators to aid in siting catheters 102 via muscle twitch response, electrical blocking of nerve and motor function, or stimulation therapy. Additionally, piezoelectric sensing may be utilized to track catheter movement (see FIG. 36).

Other manufacturing methods can involve fabricating catheter 102 constructs via injection molding or additive manufacturing techniques.

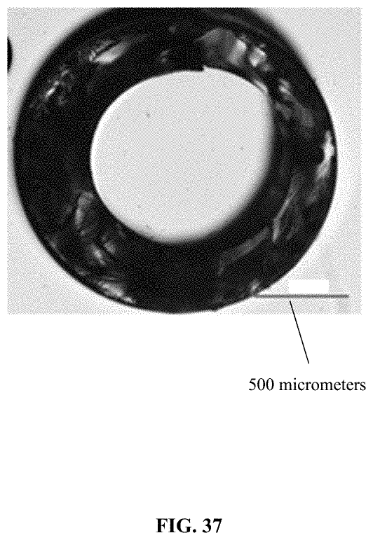

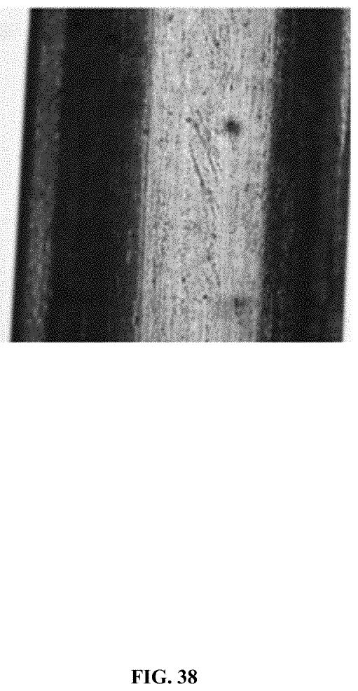

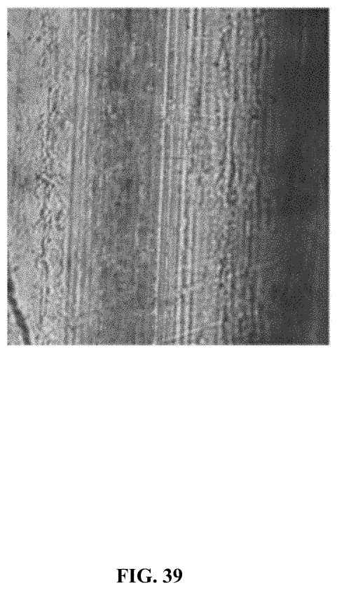

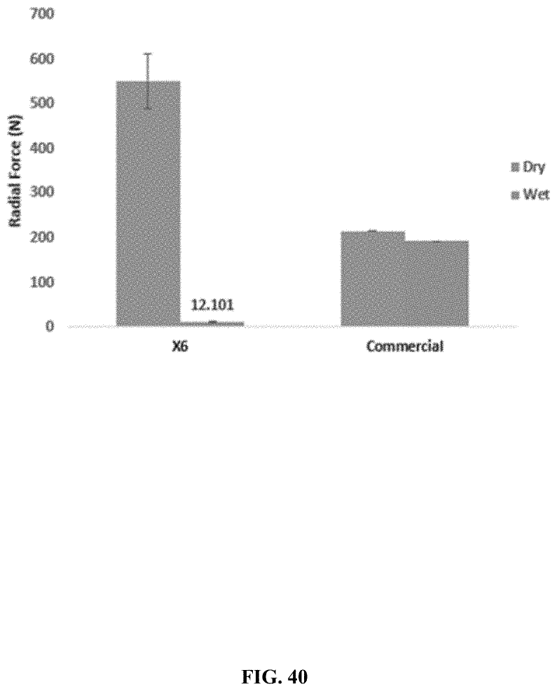

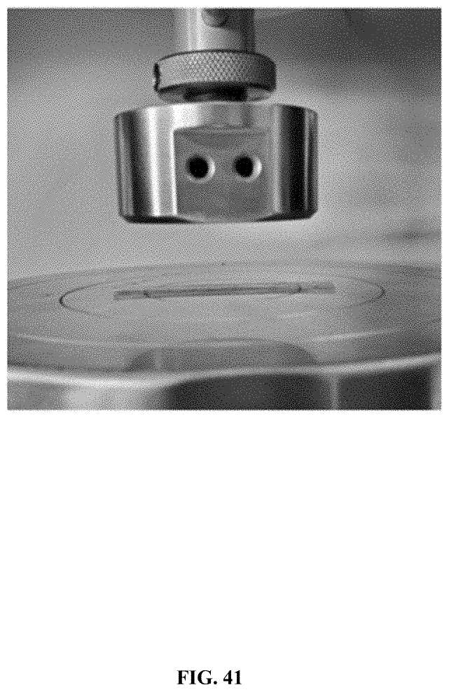

CXBE prepolymers may be utilized in the fabrication of kink resistant catheters 102 as described above (see FIGS. 23-25). Light microscopy revealed a defined lumen 104 with the desired diameter of 1 mm (see FIG. 37) and uniform structure free of defects (see FIGS. 38-39). Fabricated catheters 102, when subjected to radial compression to 50% lumen 104 reduction based on a well-established method used to test cardiac stents and other implants, achieve significantly higher radial forces (˜500N vs ˜200N for commercial catheters) in dry condition and a significant radial force of ˜12N when hydrated (comparing favorably with the desirable radial forces of ˜2 and 10N for cardiac stents) (see FIGS. 40-41). Of note, the significantly higher dry force of CXBE catheters vs. commercial catheters is advantageous in reducing the likelihood of kinking or breakage during initial insertion, while the lower wet radial force indicates that CXBE catheters 102 become softer and more compliant in the physiological environment, minimizing risk of irritation or injury of the surrounding tissue compared to commercial catheters (wet force ˜200N).

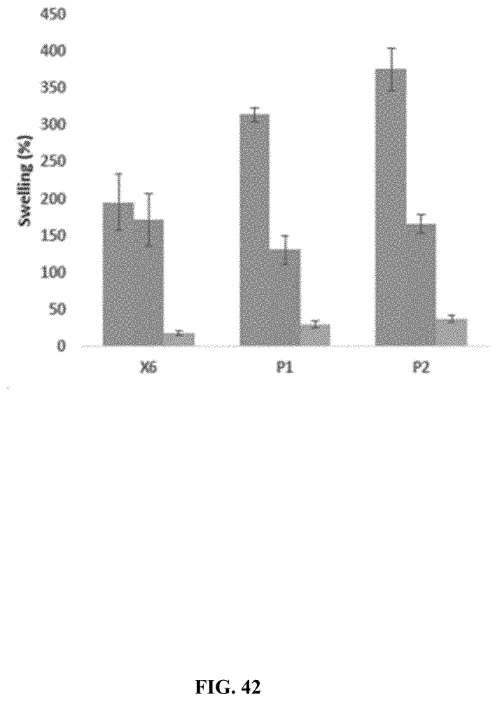

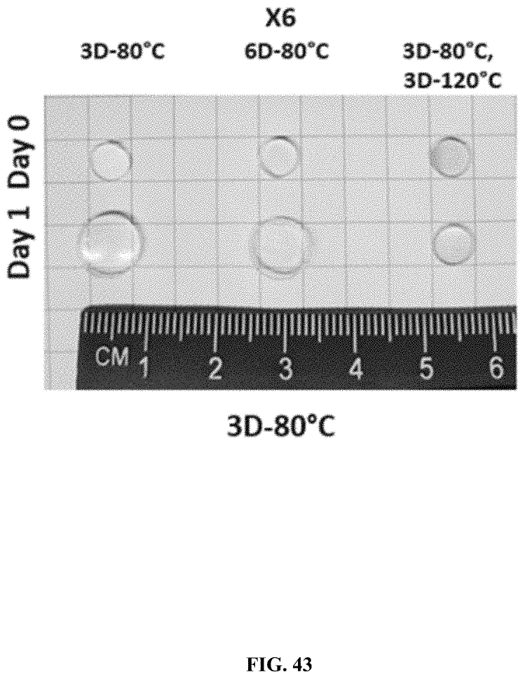

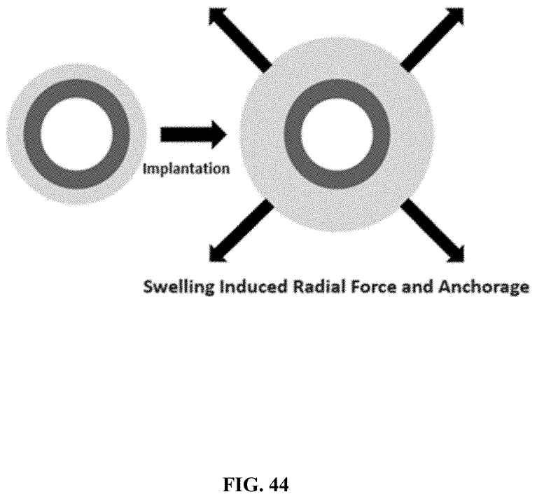

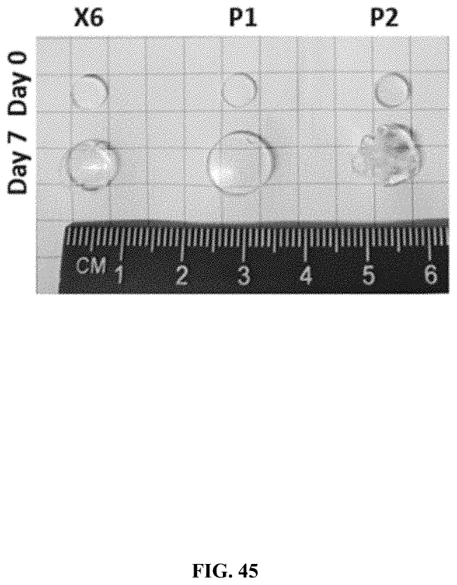

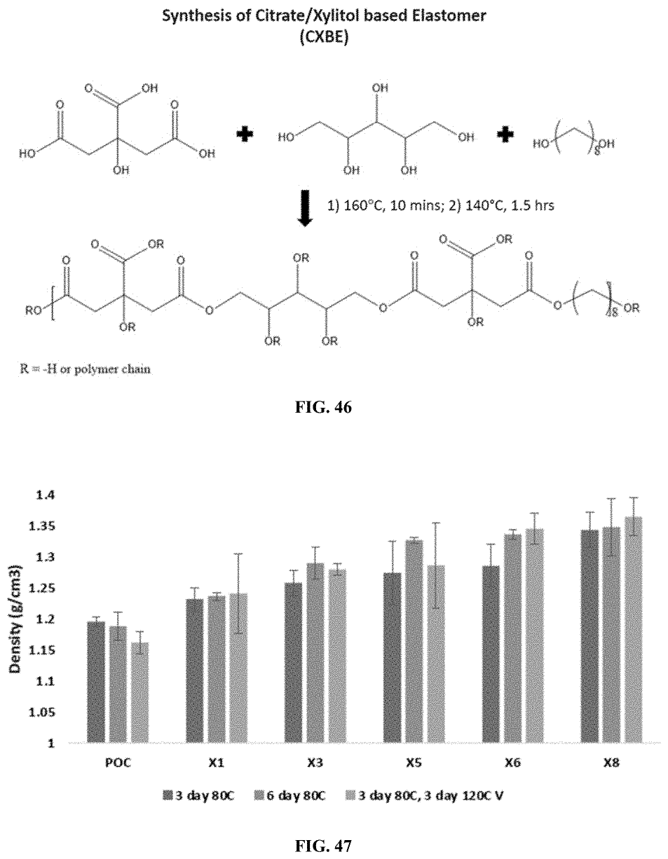

CXBEs display considerably tunable swelling upon hydration in PBS, depending on formulation and crosslinking condition (see FIG. 42). While lightly crosslinked CXBEs display large degrees of swelling by mass and volume (see FIG. 43), highly crosslinked CXBEs display minimal swelling. This property is advantageous in the design of catheters 102 with multiple phases in which the outer phase is highly swelling, generating radial force and thus anchorage in the surrounding tissue while the inner phase swells minimally, maintaining structural integrity and the internal lumen 104 (see FIG. 44). Additionally, the variable swelling rates of the CXBE polymers by day 7 (see FIG. 45) indicate tunable drug release and degradation rates (P2 appears to degrade significantly within 7 days).

In some embodiments, CXBE bisphasic catheters 102 can be fabricated with an inner low swelling phase and an outer high swelling phase (see FIG. 4) in which the outer phase provides tissue anchorage as described above. It is contemplated for catheters 102 to be fabricated with a variety of layers with variable swelling rates.

One of the benefits of the catheter 102 is its ability to function as a system; and not just a catheter. The catheter 102 is able to deliver the medications very close to the nerve or tissue intended to, by being attached or anchored to avoid any movement up to 2 months and after the completion of the therapy will biodegrade. While undergoing biodegradation to be absorbed, the sensors discussed herein that are imbedded therein can send signals to confirm the integrity/position of the catheter 102. Additionally, the sensors may stimulate currents to activate other therapeutic mechanisms. After completion of the therapy, the entire system (catheter and sensors) will biodegrade to be absorbed by the body.

The material used to fabricate the catheter 102 is made from a polymer or oligomer composition. Embodiments of the composition are described in the following sections.

In one aspect, a composition is provided comprising a polymer or oligomer formed from one or more monomers of Formula (A1), one or more monomers independently selected from Formula (B1) and Formula (B2), and one or more monomers of Formula (C1):

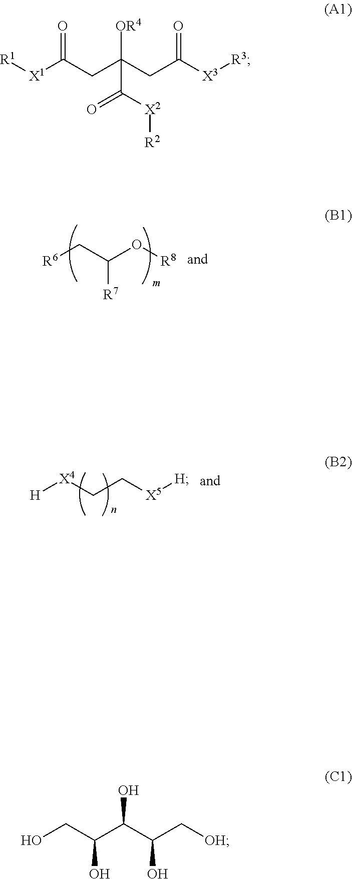

wherein:
X¹, X², and X³ are each independently —O— or —NH—;
X⁴ and X⁵ are independently —O— or —NH;
R¹, R², and R³ are each independently —H, C₁-C₂₂ alkyl, C₂-C₂₂ alkenyl, or M⁺;
R⁴ is H or M⁺;
R⁶ is —H, —OH, —OCH₃, —OCH₂CH₃; —CH₃, or —CH₂CH₃;
R⁷ is —H, C₁-C₂₃ alkyl, or C₂-C₂₃ alkenyl;
R⁸ is —H, C₁-C₂₃ alkyl, C₂-C₂₃ alkenyl, —CH₂CH₂OH, or —CH₂CH₂NH₂;
n and m are independently integers ranging from 1 to 2000; and
M⁺ is a cation.

In some embodiments, X¹, X², and X³ are each —O—. In some embodiments, R⁴ is —H. In some embodiments, the one or more monomers of Formula (A1) comprise citric acid or a citrate. In some embodiments, the one or more monomers of Formula (B1) are selected from poly(ethylene glycol) or poly(propylene glycol). In some embodiments, the one or more monomers of Formula (B2) are selected from 1,2-ethylene glycol, 1,3-propanediol, 1,4-butanediol, 1,5-pentanediol, 1,6-hexanediol, 1,8-octanediol, 1,10-decanediol, and 1,12-dodecanediol.

In some embodiments, the one or more monomers independently selected from Formula (B1) and Formula (B2) and the one or more monomers of Formula (C2) are present in a molar ratio ranging from about 20:1 to about 1:20.

In some embodiments, the polymer or oligomer is further formed from one or more monomers of Formula (D1):

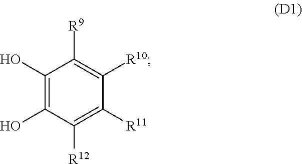

wherein:
R⁹, R¹⁰, R¹¹ and R¹² are each independently selected from —H, —OH, —CH₂(CH₂)ₓNH₂, —CH₂(CHR¹³)NH₂, —CH₂(CH₂)ₓOH, —CH₂(CHR¹³)OH, and —CH₂(CH₂)ₓCOOH;
R¹³ is —COOH or —(CH₂)_{y}COOH; and
x and y are independently an integer ranging from 1 to 10.

In some embodiments, the one or more monomers of Formula (D1) are selected from dopamine, L-DOPA, D-DOPA, gallic acid, caffeic acid, 3,4-dihydroxyhydrocinnamic acid, or tannic acid.

In some embodiments, the polymer or oligomer is further formed from one or more monomers independently selected from Formula (E1), Formula (E2), Formula (E3), and Formula (E4):

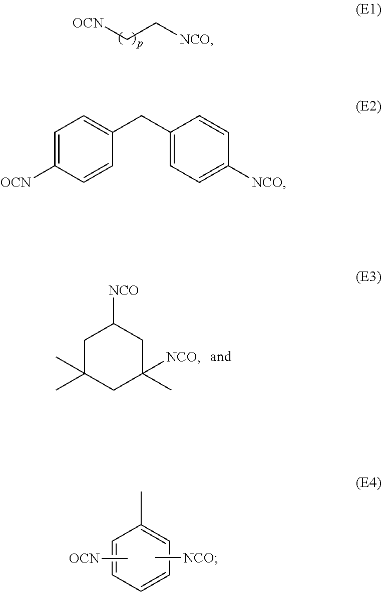

wherein p is an integer ranging from 1 to 20.

In some embodiments, the polymer or oligomer is further formed from one or more monomers independently selected from Formula (F1) or Formula (F2):

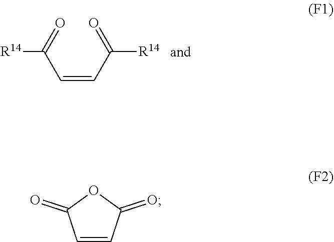

wherein R¹⁴ is selected from —OH, —OCH₃, —OCH₂CH₃, or —Cl.

In some embodiments, the polymer or oligomer is further formed from one or more monomers independently selected from Formula (G1):

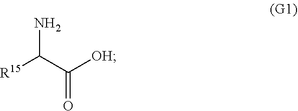

wherein R¹⁵ is an amino acid side chain.

In some embodiments, the polymer or oligomer is further formed from one or more monomers independently selected from Formula (H1), Formula (H2), and Formula (H3):

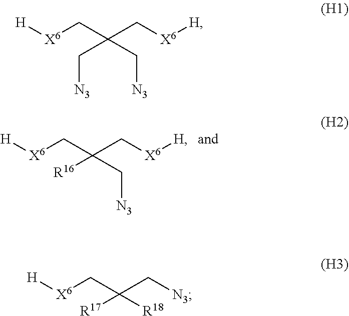

wherein:

X⁶ is independently selected at each occurrence from —O— or —NH—;

R¹⁶ is —CH₃ or —CH₂CH₃; and
R¹⁷ and R¹⁸ are each independently —CH₂N₃, —CH₃, or —CH₂CH₃.

In some embodiments, the polymer or oligomer is further formed from one or more monomers independently selected from Formula (I1), Formula (I2), Formula (I3), Formula (I4), Formula (I5), and Formula (I6):

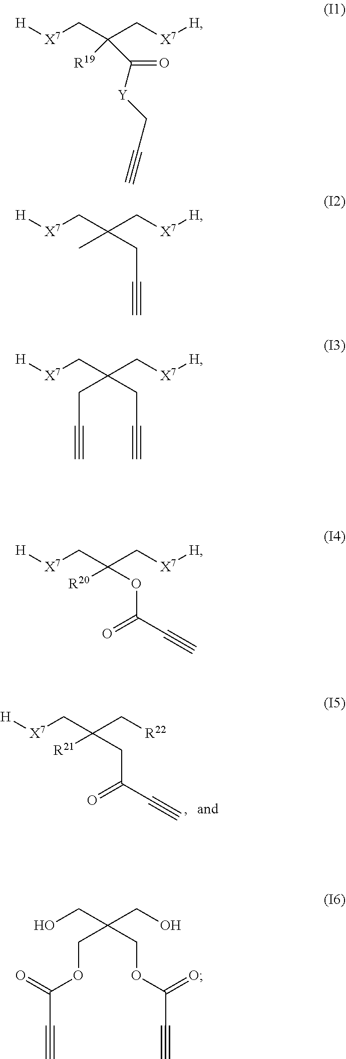

wherein:
X⁷ and Y are independently —O— or —NH—;
R¹⁹ and R²⁰ are each independently —CH₃ or —CH₂CH₃;
R²¹ is —OC(O)CCH, —CH₃, or —CH₂CH₃; and
R²² is —CH₃, —OH, or —NH₂.

In some embodiments, the polymer or oligomer is thermally crosslinked. In some embodiments, the polymer or oligomer has a cross-linking density ranging from about 600 to about 70,000 mol/m³.

In some embodiments, the composition has a tensile strength of about 1 MPa to about 120 MPa in a dry state. In some embodiments, the composition has a tensile modulus of about 1 mPA to about 3.5 GPa in a dry state. In some embodiments, the composition is luminescent.

In some embodiments, the composition further comprises an inorganic material. In some embodiments, the inorganic material is a particulate inorganic material. In some embodiments, the inorganic material is selected from hydroxyapatite, tricalcium phosphate, biphasic calcium phosphate, bioglass, ceramic, magnesium powder, pearl powder, magnesium alloy, and decellularized bone tissue particles. In such embodiments, the composition has a compressive strength ranging from about 250 MPa to about 350 MPa. In such embodiments, the composition has a compressive modulus ranging from about 100 KPa to about 1.8 GPa. In such embodiments, the composition displays room-temperature phosphorescence.

In some embodiments, the composition further comprises an antioxidant, pharmaceutically active agent, biomolecule, or cell.

In some embodiments, the composition is configured to degrade in less than 4 months.

In another aspect, a method of preparing a composition is provided comprising: polymerizing a polymerizable composition to form a polymer composition, the polymerizable composition comprising one or more monomers of Formula (A1), one or more monomers independently selected from Formula (B1) and Formula (B2), and one or more monomers of Formula (C1):

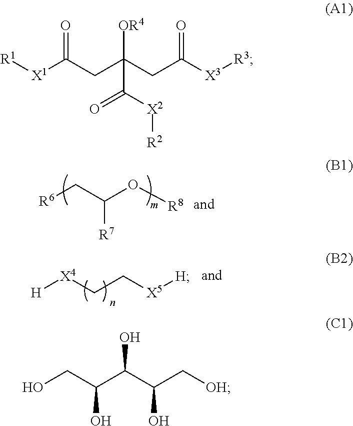

wherein all variables are as defined herein.

Some embodiments of the compositions contain citrate polymers doped with xylitol. Xylitol is an FDA approved sugar alcohol that is currently used as an alternative sweetener as well as a cavity-preventing dental rinse. Xylitol contains five hydroxyl groups capable of reacting with the carboxyl group (or derivatives thereof) of citric acid or citrate derivatives. The presence of these hydroxyl groups not only allows xylitol to be incorporated into citrate-containing polymers via esterification during polymerization, but the large number of said groups also increases the number of chemical crosslinks formed. These additional crosslinks improve the mechanical strength of the polymer, particularly the modulus. In addition, the large number of hydroxyl groups found within the xylitol monomers are capable of ionic binding with calcium, either within hydroxyapatite or deposited from an outside source. This binding improves the interface between hydroxyapatite and the polymer within compositions and increases the amount of calcium and subsequent mineral deposition in the composite surface. Previous studies conducted on rats demonstrated that oral administration of xylitol increased femur mineral density as a result of increased calcium bioavailability. Further, studies have also shown significant antibacterial and antioxidant activity of xylitol. Compared to the polyols used previously in citrate-based polymers, xylitol is more biocompatible and has increased hydrophilicity, which increases the water uptake into the polymer and/or composites and increases the rate of hydrolysis. The compositions of the present disclosure show increased mechanical properties while showing modulated degradation rates from approximately 1 year to 4 months. Therefore, the presently disclosed compositions are a system where high mechanical strength is maintained independent of biodegradation rate.

As used herein, the term “substituted” means that a hydrogen atom is removed and replaced by a substituent. It is contemplated to include all permissible substituents of organic compounds. As used herein, the phrase “optionally substituted” means unsubstituted or substituted. It is to be understood that substitution at a given atom is limited by valency. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with a permitted valence of the substituted atom and the substituent and that the substitution results in a stable compound, e.g., a compound that does not spontaneously undergo transformation such as by rearrangement, cyclization, elimination, etc. In still further aspects, it is understood that when the disclosure describes a group being substituted, it means that the group is substituted with one or more (i.e., 1, 2, 3, 4, or 5) groups as allowed by valence selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aliphatic” as used herein refers to a nonaromatic hydrocarbon group and includes branched and unbranched, alkyl, alkenyl, or alkynyl groups. As used herein, the term “Cₙ-Cₘ alkyl,” employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, heptyl, octyl, nonyl, decyl, dodecyl, tetradecyl, hexadecyl, eicosyl, tetracosyl, and the like. The alkyl group can also be substituted or unsubstituted. Throughout the specification, the term “alkyl” is generally used to refer to both unsubstituted alkyl groups and substituted alkyl groups; however, substituted alkyl groups are also specifically referred to herein by identifying the specific substituent(s) on the alkyl group. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

As used herein, “Cₙ-Cₘ alkenyl” refers to an alkyl group having one or more double carbon-carbon bonds and having n to m carbons. Examples of alkenyl groups include, but are not limited to, ethenyl, n-propenyl, isopropenyl, n-butenyl, seobutenyl, and the like. In various aspects, the alkenyl moiety contains 2 to 6, 2 to 4, or 2 to 3 carbon atoms. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, cyano, silyl, sulfo-oxo, sulfonyl, sulfone, sulfoxide, thiol, or phosphonyl, as described below.

The terms “amine” or “amino” as used herein are represented by the formula —NR^{x}R^{y}, where R^{x} and R^{y} can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NR^{x}R^{y}.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)R^{z} or C(O)OR^{z}, where R^{z} can be an alkyl, halogenated alkyl, alkenyl, alkynyl, aryl, heteroaryl, cycloalkyl, cycloalkenyl, heterocycloalkyl, or heterocycloalkenyl group described above.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within the second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the endpoints of the range unless expressly stated otherwise. For example, a range of “between 5 and 10,” “from 5 to 10,” or “5-10” should generally be considered to include the endpoints 5 and 10. Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

As used herein, the term “composition” is intended to encompass a product comprising the specified ingredients in the specified amounts, as well as any product which results, directly or indirectly, from a combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

It will be understood that, although the terms “first,” “second,” etc., may be used herein to describe various elements, components, regions, layers and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the terms “substantially identical reference composition” refers to a reference composition comprising substantially identical components in the absence of an inventive component. In another exemplary aspect, the term “substantially” in, for example, the context “substantially identical reference composition” refers to a reference composition comprising substantially identical components and wherein an inventive component is substituted with a component common in the art.

While aspects of the present invention can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Compositions

In one aspect, a composition is provided comprising a polymer or oligomer formed from one or more monomers of Formula (A1), one or more monomers independently selected from Formula (B1) and Formula (B2), and one or more monomers of Formula (C1):

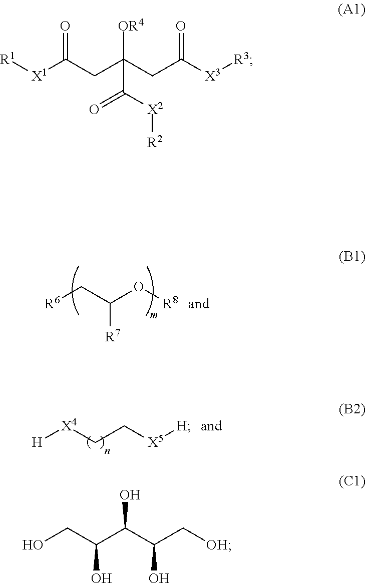

wherein:
X¹, X², and X³ are each independently —O— or —NH—;
X⁴ and X⁵ are independently —O— or —NH;
R¹, R², and R³ are each independently —H, C₁-C₂₂ alkyl, C₂-C₂₂ alkenyl, or M⁺;
R⁴ is H or M⁺;
R⁶ is —H, —OH, —OCH₃, —OCH₂CH₃; —CH₃, or —CH₂CH₃;
R⁷ is —H, C₁-C₂₃ alkyl, or C₂-C₂₃ alkenyl;
R⁸ is —H, C₁-C₂₃ alkyl, C₂-C₂₃ alkenyl, —CH₂CH₂OH, or —CH₂CH₂NH₂;
n and m are independently integers ranging from 1 to 2000; and
M⁺ is a cation.

In some embodiments, X¹ is —O—. In some embodiments, X² is —O—. In some embodiments, X³ is —O—. In some embodiments, X¹, X², and X³ are each —O—.

In some embodiments, X⁴ is —O. In some embodiments, X⁴ is —NH—. In some embodiments, X⁵ is —O—. In some embodiments, X⁵ is —NH—. In some embodiments, X⁴ and X⁵ are each —O—. In some embodiments, X⁴ and X⁵ are each —NH—. In some embodiments, one of X⁴ and X⁵ is —O— and the other of X⁴ and X⁵ is —NH—.

In some embodiments, R¹, R², and R³ are each independently —H, —CH₃, or —CH₂CH₃.

In some embodiments, R¹, R² and R³ are each independently —H or M⁺.

In some embodiments, R⁴ is —H.

In some embodiments, R⁴ is M⁺.

In some embodiments, M⁺ is independently at each occurrence Na⁺ or K⁺.

In some embodiments, R⁶ is —OH.

In some embodiments, R⁷ is —H. In some embodiments, R⁷ is —CH₃.

In some embodiments, R⁸ is —H.

In some embodiments, n and m can independently be an integer from 1 to 2000, including exemplary values of 1 to 100, or 1 to 250, or 1 to 500, or 1 to 750 or 1 to 1000, or 1 to 1250, or 1-1500, or 1 to 1750. In yet other aspects, n and m can independently be an integer between 1 and 20, including exemplary values of 2, 3, 4, 5, 6, 7, 8, 9, 10, 11, 12, 13, 14, 15, 16, 17, 18, and 19.

In some embodiments, the one or more monomers of Formula A1 can comprise an alkoxylated, alkenoxylated, or non-alkoxylated and non-alkenoxylated citric acid, citrate, or ester or amide of citric acid.

In some embodiments, the one or more monomers of Formula B1 are selected from poly(ethylene glycol) (PEG) or poly(propylene glycol) (PPG) having terminal hydroxyl or amine groups. Any such PEG or PPG not inconsistent with the objected of the present disclosure may be used. In some embodiments, for example, a PEG or PPG having a weight average molecular weight between about 100 and about 5000 or between about 200 and about 1000 or between 200 and about 100,000 may be used.

In some embodiments, the one or more monomers of Formula B2 may comprise C₂-C₂₀, C₂-C₁₂, or C₂-C₆ aliphatic alkane diols or diamines. For instance, the one or more monomers of Formula B2 may comprise 1,4-butanediol, 1,4-butanediamine, 1,6-hexanediol, 1,6-hexanediamine, 1,8-octanediol, 1,8-octanediamine, 1,10-decanediol, 1,10-decanediamine, 1,12-dodecanediol, 1,12-dodecanediamine, 1,16-hexadecanediol, 1,16-hexadecanediamine, 1,20-icosanediol, or 1,20-icosanediamine. In alternative embodiments, the one or more monomers of Formula B2 may be replaced by a branched alkanediol/diamine, alkenediol/diamine, or an aromatic diol/diamine.

In some embodiments, the polymer may be formed from a molar ratio of the one or more monomers of Formula (A1) to the one or more monomers of Formula (B1), Formula B2), and Formula (C1) [A1:(B1+B2+C1)] ranging from about 3:1 to about 1:3, for example about 3:1, about 2.5:1, about 2:1, about 1.5:1, about 1:1, about 1:1.5, about 1:2, about 1:2.5, or about 1:3.

In some embodiments, the polymer or oligomer may further be formed from one or more monomers comprising a catechol-containing species. The catechol containing species can comprise any catechol-containing species not inconsistent with the objects of the present disclosure. In some cases, a catechol-containing species comprises at least one moiety that can form an ester or amide bond with another chemical species used to form a polymer in embodiments were the monomers are reacted. For example, in some embodiments, a catechol-containing species comprises an alcohol moiety, an amine moiety, a carboxylic acid moiety, or combinations thereof. Further, in some embodiments, a catechol-containing species comprises a hydroxyl moiety that is not part of the catechol moiety. In some embodiments, a catechol-containing species comprises dopamine. In other embodiments, a catechol-containing species comprises L-3,4-dihydroxyphenylalanine (L-DOPA) or D-3,4-dihydroxyphenylalanine (D-DOPA). In still other embodiments, a catechol-containing species comprises gallic acid or caffeic acid. In some embodiments, a catechol-containing species comprises 3,4-dihydroxycinnamic acid. Additionally, a catechol-containing species may also comprise a naturally-occurring species or a derivative thereof, such as tannic acid or a tannin. Moreover, in some embodiments, a catechol-containing species is coupled to the backbone of the polymer or oligomer through an amide bond. In other embodiments, a catechol-containing species is coupled to the backbone of the polymer or oligomer through an ester bond. Further examples of catechol-containing species can be found in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers of Formula (D1):

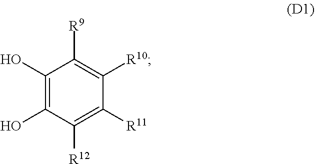

wherein:
R⁹, R¹⁰, R¹¹ and R¹² are each independently selected from —H, —OH, —CH₂(CH₂)ₓNH₂, —CH₂(CHR¹³)NH₂, —CH₂(CH₂)ₓOH, —CH₂(CHR¹³)OH, and —CH₂(CH₂)^{x}COOH;
R¹³ is —COOH or —(CH₂)_{y}COOH; and
x and y are independently an integer ranging from 1 to 10.

In some embodiments, the one or more monomers of Formula (D1) are selected from dopamine, L-DOPA, D-DOPA, gallic acid, caffeic acid, 3,4-dihydroxyhydrocinnamic acid, or tannic acid.

In some embodiments, the polymer or oligomer may further be formed from one or more monomers comprising a diisocyanate. In some embodiments, an isocyanate comprises an alkane diisocyanate having four to twenty carbon atoms. An isocyanate described herein may also include a monocarboxylic acid moiety. Further examples of various isocyanates which can be used are described in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers independently selected from Formula (E1), Formula (E2), Formula (E3), and Formula (E4):

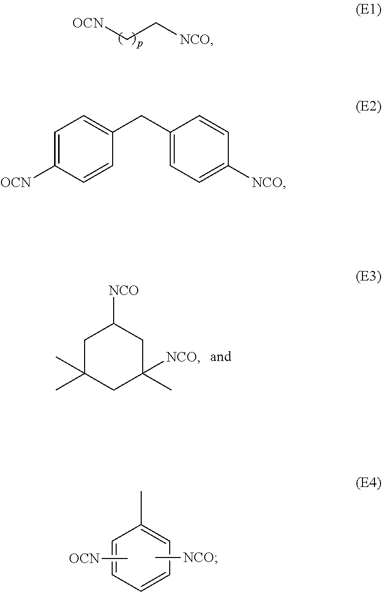

wherein p is an integer ranging from 1 to 20.

In some embodiments, the polymer or oligomer may further be formed from one or more monomers comprising a polycarboxylic acid, such as a dicarboxylic acid, or a functional equivalent of a polycarboxylic acid, such as a cyclic anhydride or an acid chloride of a polycarboxylic acid. In some embodiments, the polycarboxylic acid or functional equivalent thereof can be saturated or unsaturated. In some embodiments, for example, the polycarboxylic acid or functional equivalent thereof comprises maleic acid, maleic anhydride, fumaric acid, or fumaryl chloride. In some embodiments, a vinyl-containing polycarboxylic acid or functional equivalent thereof may also be used, such as allylmalonic acid, allylmalonic chloride, itaconic acid, or itaconic chloride. Further, in some embodiments, the polycarboxylic acid or functional equivalent thereof can be at least partially replaced with an olefin-containing monomer that may or may not be a polycarboxylic acid. In some embodiments, for instance, an olefin-containing monomer comprises an unsaturated polyol such as a vinyl-containing diol. Further examples can be found in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers independently selected from Formula (F1) or Formula (F2):

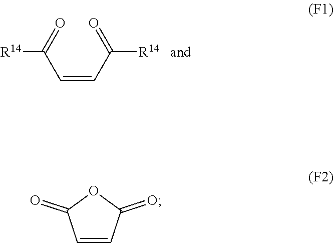

wherein R¹⁴ is selected from —OH, —OCH₃, —OCH₂CH₃, or —Cl.

In some embodiments, the polymer or oligomer may further be formed from one or more monomers comprising an amino acid, such as an alpha-amino acid. An alpha-amino acid of a polymer described herein, in some embodiments, comprises an L-amino acid, a D-amino acid, or a D,L-amino acid. In some embodiments, an alpha-amino acid comprises alanine, arginine, asparagine, aspartic acid, cysteine, glycine, glutamine, glutamic acid, histidine, isoleucine, leucine, lysine, methionine, proline, phenylalanine, serine, threonine, tyrosine, tryptophan, valine, or a combination thereof. Further, in some embodiments, an alpha-amino acid comprises an alkyl-substituted alpha-amino acid, such as a methyl-substituted amino acid derived from any of the 22 “standard” or proteinogenic amino acids, such as methyl serine.

In some embodiments, the polymer or oligomer may further be formed from one or more monomers independently selected from Formula (G1):

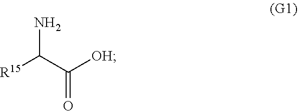

wherein R¹⁵ is an amino acid side chain.

In some embodiments, the polymer or oligomer may further be formed from one or more monomers comprising one or more alkyne moieties and/or one or more azide moieties. The monomer comprising one or more alkyne and/or azide moieties used to form a polymer described herein can comprise any alkyne- and/or azide-containing chemical species not inconsistent with the objectives of the present disclosure. Additional examples of monomers containing alkyne and/or azide moieties can be found in U.S. Patent Application Publication No. 2020/0140607 and International Patent Application Publication No. WO2018/227151, the contents of which are incorporated herein in their entirety.

In some embodiments, the polymer or oligomer may further be formed from one or more monomers independently selected from Formula (H1), Formula (H2), and Formula (H3):

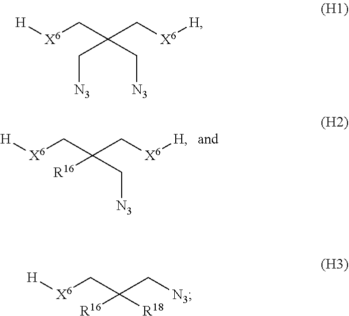

wherein:
X⁶ is independently selected at each occurrence from —O— or —NH—;
R¹⁶ is —CH₃ or —CH₂CH₃; and
R¹⁷ and R¹⁸ are each independently —CH₂N₃, —CH₃, or —CH₂CH₃.

In some embodiments, the polymer or oligomer may further be formed from one or more monomers independently selected from Formula (I1), Formula (I2), Formula (I3), Formula (I4), Formula (I5), and Formula (I6):

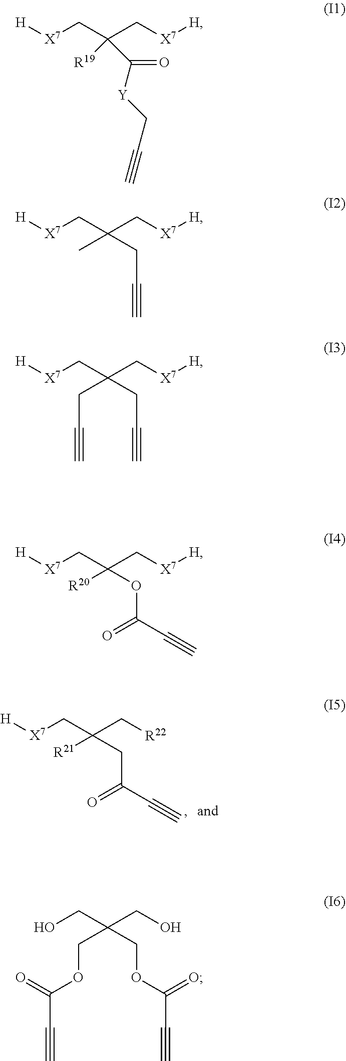

wherein:
X⁷ and Y are independently —O— or —NH—;
R¹⁹ and R²⁰ are each independently —CH₃ or —CH₂CH₃;
R²¹ is —OC(O)CCH, —CH₃, or —CH₂CH₃; and
R²² is —CH₃, —OH, or —NH₂.

In some embodiments, a monomer described herein can be functionalized with a bioactive species. Moreover, said monomer can comprise one or more alkyne and/or azide moieties. For example, in some embodiments, a polymer or oligomer described herein is formed from one or more monomers containing a peptide, polypeptide, nucleic acid, or polysaccharide, wherein the peptide, polypeptide, nucleic acid, or polysaccharide is functionalized with one or more alkyne and/or azide moieties. In some embodiments, the bioactive species described herein is a growth factor or signaling molecule. Further, the peptide can comprise a dipeptide, tripeptide, tetrapeptide, or a longer peptide.

In some embodiments, the stoichiometric ratio of carboxylic acid groups or derivatives thereof to hydroxyl groups within the monomers used to form the polymer or oligomer is about 1:1. In some embodiments, the stoichiometric ratio of carboxylic acid groups or derivatives thereof to hydroxyl groups within the monomers used to form the polymer or oligomer is less than about 1:1. If the stoichiometric ratio is less than about 1:1, the polymer or oligomer may show defined regions of hydrogen bonding. A composition described herein, in some cases, is a condensation polymerization reaction product of the identified species. Thus, in some embodiments, at least two of the identified species are co-monomers for the formation of a copolymer. In some such embodiments, the reaction product forms an alternating copolymer or a statistical copolymer of the co-monomers. Additionally, as described further herein, species described herein may also form pendant groups or side chains of a copolymers.

Additionally, in some embodiments, a composition comprising a polymer described herein can further comprise a crosslinker. Any crosslinker not inconsistent with the objectives of the present disclosure may be used. In some cases, for example, a crosslinker comprises one or more olefins or olefinic moieties that can be used to crosslink polymers containing ethylenically unsaturated moieties. In some embodiments, a crosslinker comprises an acrylate or polyacrylate, including a diacrylate. In other embodiments, a crosslinker comprises one or more of 1,3-butanediol diacrylate, 1,6-hexanediol diacrylate, glycerol 1,3-diglyerolate diacrylate, d(ethylene glycol) diacrylate, poly(ethylene glycol) diacrylate, poly(propylene glycol) diacrylate, and propylene glycol glycerolate diacrylate. In still other embodiments, a crosslinker comprises a nucleic acid, including DNA or RNA. In still other instances, a crosslinker comprises a “click chemistry” reagent, such as an azide or an alkyne. In some embodiments, a crosslinker comprises an ionic crosslinker. For instance, in some embodiments, a polymer is crosslinked with a multivalent metal ion, such as a transition metal ion. In some embodiments, a multivalent metal ion used as a crosslinker of the polymer comprises one or more of Fe, Ni, Cu, Zn, or Al, including in the +2 or +3 state.

In addition, a crosslinker described herein can be present in a composition in any amount not inconsistent with the objective of the present disclosure. For example, in some embodiments, a crosslinker is present in a composition in an amount between about 5 weight percent and about 50 weight percent, between about 5 weight percent and about 40 weight percent, between about 5 weight percent and about 30 weight percent, between about 10 weight percent and about 40 weight percent, between about 10 weight percent and about 30 weight percent, or between about 20 weight percent and about 40 weight percent, based on the total weight of the composition.

Thus, in some embodiments, the composition described herein comprises a polymer described herein that is crosslinked to from a polymer network. In some embodiments, the polymer network comprises a hydrogel. A hydrogel, in some cases, comprises an aqueous continuous phase and polymeric disperse or discontinuous phase. Further in some embodiments, the crosslinked polymer network described herein is not water soluble.

Such a polymer network can have a high cross-linking density. “Cross-linking density”, for reference purposes herein, can refer to the number of cross-links between polymer backbones or the molecular weight between cross-linking sites. Cross-links may include, for example, ester bonds formed by the esterification or reaction of one or more pendant carboxyl or carboxylic acid groups with one or more pendant hydroxyl groups of adjacent polymer backbones. In some embodiments, a polymer network described herein has a cross-linking density of at least about 500, at least about 1000, at least about 5000, at least about 7000, at least about 10,000, at least about 20,000, or at least about 30,000 mol/m³. In some embodiments, the cross-linking density is between about 600 and about 70,000, or between about 10,000 and about 70,000 mol/m³.

In some embodiments, the compositions described herein show decreased molecular weight and increasing crosslink density as compared to a substantially identical reference composition not formed from a monomer of Formula (C1).

In some embodiments, the compositions described herein show increased hydrophilicity as compared to a substantially identical reference composition not formed from a monomer of Formula (C1).

In some embodiments, the compositions described herein show increased fluorescence as compared to a substantially identical reference composition not formed from a monomer of Formula (C1).

In some embodiments, the compositions described herein can exhibit a tensile strength of about 1 MPa to about 120 MPa in a dry state as measured according to ASTM Standard D412A, for example of about 2 MPa, 10 MPa, 20 MPa, 30 MPa, 40 MPa, 50 MPa, 60 MPa, 70 MPa, 80 MPa, 90 MPa, or 100 MPa.

In some embodiments, the compositions described herein can exhibit a tensile modulus of about 11\4 Pa to about 3.5 GPa in a dry state as measured according to ASTM Standard D412A, for example about 11\4 Pa, about 10 MPa, about 50 MPa, about 100 MPa, about 250 MPa, about 500 MPa, about 750 MPa, about 1 GPa, about 1.5 GPa, about 2 GPa, about 2.5 GPa, about 3 GPa, or about 3.5 GPa.

Various components of compositions which may form part or all of a catheter 102 utilized have been described herein. It is to be understood that a composition according to the present disclosure can comprise any combination of components and features not inconsistent with the objectives of the present disclosure. For example, in some cases, a composition forming part or all of a catheter 102 utilized in a composition described herein can comprise a combination, mixture, or blend of polymers described herein. Additionally, in some embodiments, such a combination, mixture, or blend can be selected to provide a catheter 102 having any biodegradability, mechanical property, and/or chemical functionality described herein.

Further, one or more polymers described herein can be present in a composition forming part or all of a catheter 102 utilized in any amount not inconsistent with the objectives of the present disclosure. In some embodiments, a catheter consists or consists essentially of the one or more polymers described herein. In other instances, a catheter 102 comprises up to about 95 weight percent, up to about 90 weight percent, up to about 80 weight percent, up to about 70 weight percent, up to about 60 weight percent, up to about 50 weight percent, up to about 40 weight percent, or up to about 30 weight percent polymer, based on the total weight of the catheter 102. In some embodiments, the balance of a catheter 102 described herein can be water, an aqueous solution, and/or an inorganic material as described further below. In some embodiments, the composition can further comprise an inorganic material. In some embodiments, the inorganic material comprises a particulate inorganic material. Any particulate inorganic material not inconsistent with the objectives of the present disclosure may be used. In some cases, the particulate inorganic material comprises one or more of hydroxyapatite, tricalcium phosphate (including alpha- and beta-tricalcium phosphate), biphasic calcium phosphate, bioglass, ceramic, magnesium powder, pearl powder, magnesium alloy, and decellularized bone tissue particle. Other particular materials may also be used.

In addition, a particular inorganic material described herein can have any particle size and/or particle shape not inconsistent with the objective of the present disclosure. In some embodiments, for instance, a particulate material has an average particle size in at least one dimension of less than about 1000 μm, less than about 800 μm, less than about 500 μm, less than about 300 μm, less than about 100 μm, less than about 50 μm, less than about 30 μm, or less than about 10 μm. In some cases, a particular material has an average particle size in at least one dimension of less than about 1 μm, less than about 500 nm, less than about 300 nm, less than about 100 nm, less than about 50 nm, or less than about 30 nm. In some instances, a particulate material has an average particle size recited herein in two dimension or three dimensions. Moreover, a particulate material can be formed of substantially spherical particles, plate-lite particles, needle-like particles, or a combination thereof. Particulate materials having other shapes may also be used.

A particular inorganic material can be present in the compositions described herein in any amount not inconsistent with the objective of the present disclosure. For example, in some cases, a composition utilized as a catheter 102 described herein comprises up to about 30 weight percent, up to about 40 weight percent, up to about 50 weight percent, up to about 60 weight percent, or up to about 70 weight percent particular materials, based on the total weight of the composition. In some instances, a composition comprises between about 1 and about 70 weight percent, between about 10 and about 70 weight percent, between about 15 and about 60 weight percent, between about 25 and about 65 weight percent, between about 26 and about 50 weight percent, between about 30 and about 70 weight percent, or between about 50 and about 70 weight percent particulate material, based on the total weight of the composition. For example, a composition described herein may comprise up to about 65 weight percent hydroxyapatite.

In some embodiments, the compositions further comprising inorganic materials can have a compressive strength as measured by ASTM Standard D695-15 of about 250 MPa to about 350 MPa, for example about 275 MPa, 300 MPa, or 325 MPa.

In some embodiments, compositions described herein further comprising inorganic materials can have a compressive modulus as measured by ASTM Standard D695-15 of about 100 KPa to about 1.8 GPa, for example about 100 kPa, about 101 MPa, about 50 Mpa, about 100 Mpa, about 250 MPa, about 500 MPa, about 750 MPa, about 1.0 GPa, about 1.2 GPa, about 1.4 GPa, about 1.6 GPa, or about 1.8 GPa.

In some embodiments, compositions described herein further comprising inorganic materials may display room temperature phosphorescence.

In another aspect, incorporation of monomer of Formula (C1) in the compositions described herein does not substantially increase swelling of the composite material.

In some embodiments, the catheter 102 described herein is a polymer network. The polymer network can comprise any combination of polymers and/or copolymers described above. Further, in some embodiments, the polymer network comprises an inorganic material (such as a particulate inorganic material). For example, polymers as described above can be cross-linked to encapsulate or otherwise bond to the inorganic material. Cross-linking can be performed, for example, by exposing the polymer to heat and/or UV light.

In other embodiments, the composition described herein can have additional desirable properties suitable for use in methods described herein. In some embodiments, the composition is luminescent. In some cases, such luminescence is photoluminescence and can be observed by exposing the composition to suitable wavelength of light, such as light having a peak or average wavelength between 400 nm and 600 nm. Moreover, in some embodiments, the luminescence intensity of the composition, measured in arbitrary or relative units, can be used as a measure of degradation of the catheter 102 over time, thereby indicating biodegradability or clearance from a site.

In some embodiments, the compositions described herein deliver citrate and xylitol to the site of action due to their release upon degradation of the composition. In some embodiments, release of xylitol and citrate may enhance differentiation and tissue regeneration. In some embodiments, release of xylitol may increase tissue regeneration by enhancing bioavailability of calcium. In some embodiments, release of xylitol exerts antioxidant and anti-inflammatory action on surrounding cells and/or tissues. In some embodiments, release of xylitol and citrate may exert an antimicrobial effect such that it prevents local or implant-associated infection.

### Methods of Preparation

Further provided are methods of preparing the compositions as described hereinabove. In one aspect, a method is provided for preparing a composition as described herein comprising polymerizing a polymerizable composition comprising:

one or monomers of Formula (A1):

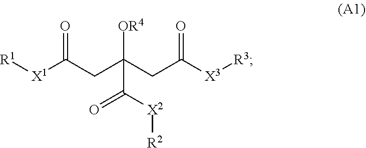

one or more monomers independently selected from Formula (B1) and Formula (B2):

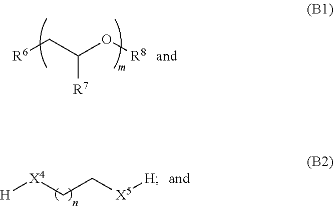

and and one or more monomers of Formula (C1):

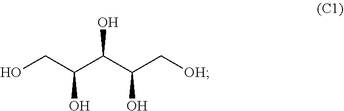

to form a polymer;
wherein:
X¹, X², and X³ are each independently —O— or —NH—;
X⁴ and X⁵ are independently —O— or —NH;
R¹, R², and R³ are each independently —H, C₁-C₂₂ alkyl, C₂-C₂₂ alkenyl, or M⁺;
R⁴ is H or M⁺;
R⁶ is —H, —NH, —OH, —OCH₃, —OCH₂CH₃; —CH₃, or —CH₂CH₃;
R⁷ is —H, C₁-C₂₃ alkyl, or C₂-C₂₃ alkenyl;
R⁸ is —H, C₁-C₂₃ alkyl, C₂-C₂₃ alkenyl, —CH₂CH₂OH, or —CH₂CH₂NH₂;
n and m are independently integers ranging from 1 to 2000; and
M⁺ is a cation.

In some embodiments, X¹ is —O—. In some embodiments, X² is —O—. In some embodiments, X³ is —O—. In some embodiments, X¹, X², and X³ are each —O—. In some embodiments, X⁴ is —O. In some embodiments, X⁴ is —NH—. In some embodiments, X⁵ is —O—. In some embodiments, X⁵ is —NH—. In some embodiments, X⁴ and X⁵ are each —O—. In some embodiments, X⁴ and X⁵ are each —NH—. In some embodiments, one of X⁴ and X⁵ is —O— and the other of X⁴ and X⁵ is —NH—. In some embodiments, R¹, R², and R³ are each independently —H, —CH₃, or —CH₂CH₃. In some embodiments, R¹, R² and R³ are each independently —H or M⁺. In some embodiments, R⁴ is —H. In some embodiments, R⁴ is M⁺. In some embodiments, M⁺ is independently at each occurrence Na⁺ or K⁺. In some embodiments, R⁶ is —OH. In some embodiments, R⁷ is —H. In some embodiments, R⁷ is —CH₃. In some embodiments, R⁸ is —H.

In some embodiments, n and m can independently be an integer from 1 to 2000, including exemplary values of 1 to 100, or 1 to 250, or 1 to 500, or 1 to 750 or 1 to 1000, or 1 to 1250, or 1-1500, or 1 to 1750. In yet other aspects, n and m can independently be an integer between 1 and 20, including exemplary values of 2, 3, 4, 5, 6, 7, 8, 9, 10, 11, 12, 13, 14, 15, 16, 17, 18, and 19.

In some embodiments, the one or more monomers of Formula A1 can comprise an alkoxylated, alkenoxylated, or non-alkoxylated and non-alkenoxylated citric acid, citrate, or ester or amide of citric acid.

In some embodiments, the one or more monomers of Formula B1 are selected from poly(ethylene glycol) (PEG) or poly(propylene glycol) (PPG) having terminal hydroxyl or amine groups. Any such PEG or PPG not inconsistent with the objected of the present disclosure may be used. In some embodiments, for example, a PEG or PPG having a weight average molecular weight between about 100 and about 5000 or between about 200 and about 1000 or between 200 and about 100,000 may be used.

In some embodiments, the one or more monomers of Formula B2 may comprise C₂-C₂₀, C₂-C₁₂, or C₂-C₆ aliphatic alkane diols or diamines. For instance, the one or more monomers of Formula B2 may comprise 1,4-butanediol, 1,4-butanediamine, 1,6-hexanediol, 1,6-hexanediamine, 1,8-octanediol, 1,8-octanediamine, 1,10-decanediol, 1,10-decanediamine, 1,12-dodecanediol, 1,12-dodecanediamine, 1,16-hexadecanediol, 1,16-hexadecanediamine, 1,20-icosanediol, or 1,20-icosanediamine. In alternative embodiments, the one or more monomers of Formula B2 may be replaced by a branched alkanediol/diamine, alkenediol/diamine, or an aromatic diol/diamine.

In another aspect, the method may further comprise crosslinking the polymer to provide a crosslinked polymer. The polymer may be crosslinked using any of the appropriate methods for crosslinking described herein and as would be readily apparent to those of skill in the art. In some embodiments, the polymer is crosslinked using a crosslinker. In some embodiments, crosslinking the polymer comprises thermally crosslinking the polymer.

In some embodiments, the polymer is solvent cast to form a film prior to crosslinking (such as thermal crosslinking). In other embodiments, the polymer is mixed with an inorganic material to form a homogenous mixture as described herein prior to crosslinking (such as thermal crosslinking). In some embodiments, the homogenous mixture is molded prior to crosslinking (such as thermal crosslinking).

In some embodiments, the method further comprises adding at least one biologically active agent to the formed composition.

In another aspect, this disclosure describes a method for making xylitol doped poly(octamethylene citrate) (POC) polyesters and films and composites of the same. Xylitol is incorporated into the polymer via esterification. Xylitol doped polymers can be formed into films through solvent casting followed by further crosslinking via thermal esterification, and composites via physical mixing of polymer with hydroxyapatite or other filers, molding and subsequent thermal crosslinking.

In another aspect, the compositions and methods of this disclosure incorporate xylitol homogenously into POC though chemical reaction.

In another aspect, the compositions of this disclosure increase the mechanical strength and degradation rate of POC films in dry and hydrated conditions through xylitol doping. Additionally, this compositions and methods of this disclosure tune the degradation rate of materials independently of mechanical properties through xylitol doping.

In another aspect, the compositions and methods of this disclosure fabricate catheters 102 and composites with homogenous physical properties and improved mechanical strength utilizing xylitol doped POC.

In another aspect, the compositions and methods of the disclosure fabricate materials with antibacterial capability using xylitol doped POC.

In another aspect, the compositions and methods of the disclosure fabricate materials with antioxidant and immunomodulatory capability through xylitol doping of citrate-based materials.

In another aspect, the compositions and methods of the disclosure incorporate xylitol doping into various citrate based materials including but not limited to poly(octamethylene citrate) (POC), biodegradable photoluminescent polymer (BPLPs), and injectable citrate based mussel inspired bioadhesives (iCMBAs).

In another aspect, the compositions and methods of the disclosure fabricate stimuli responsive self-healing citrate-based materials utilizing xylitol doping.

In another aspect, the compositions and methods of the disclosure create photoluminescent materials through xylitol doping of citrate-based materials.

In another aspect, the compositions and methods of the disclosure create materials with controlled and tunable release of bioactive factors (citrate and xylitol) for synergistic biological activity through xylitol doping of citrate-based materials.

Results described further herein demonstrate that varying the ratio of xylitol within POC/HA compositions provides for homogenous increases in mechanical properties while modulating the biodegradation rate significantly. Thus, incorporating of xylitol into citrate-based materials results in improved composition materials through enhanced physical and biological properties. For example, methods disclosed herein provide for homogenous incorporation of xylitol into POC via xylitol doping. The homogenous incorporation of xylitol into POC provides for compositions with increased mechanical strength and improved (quicker and more controllable) biodegradation rate, as compared to traditional POC compositions. The increased mechanical strength and improved biodegradation is exhibited in both dry and hydrated conditions. Additionally, the biodegradation rate of composite materials is tunable. It is important to note that the tunability of the biodegradation rate is independent of mechanical properties, i.e., the biodegradation rate can be tuned with little to no change in mechanical properties.

Examples methods disclosed herein involve fabricating xylitol dopes POC materials (e.g., polymers, films, catheters 102, and compositions, etc.). Polymers other than POC can be used, such a biodegradable photoluminescent polymers (BPLPs), injectable citrate-based mussel inspired bioadhesives (iCMBA), etc. Xylitol can be incorporated into the polymer via esterification. In one representative example, citric acid and octanediol/xlitol with a 1:1 mole ratio can be melted at 160° C. under stirring for ten minutes. The reaction temperature can then be reduced to 140° C., wherein the reaction proceeds until the pre-polymer can no longer be stirred due to viscosity, at which point the reaction may be quenched with dioxane. Following polymerization, the pre-polymer can be purified by precipitation in deionized water, lyophilized, and dissolved in organic solvent to form pre-polymer solutions.

Xylitol doped citrate-based polyesters may be synthesized via the above general procedure using a variety of diols. Suitable diols can be small molecule diols such as 1,2-ethylene glycol, 1,3-propanediol, 1,4-butanediol, 1,5-pentanediol, 1,6-hexanediol, 1,8-octanediol, 1,10-decanediol, and 1,12-dodecanediol or macrodiols such as poly(ethylene glycol) (PEG) or combinations thereof. Xylitol doped polymers may be synthesized with citrate:diol+xylitol ratios of 1.5:1 to 1:1.5. Xylitol doped polymers may be synthesized with varying xylitol contents from greater than 0 to less than 100% diol substitution.

Xylitol doped polymers can be formed into films through solvent casting followed by further crosslinking via thermal esterification. For instance, xylitol doped POC films can be prepared by casting prepolymer solutions in Teflon dishes, followed by solvent evaporation and thermal crosslinking.

Xylitol doped polymers can be formed into composites via physical mixing of polymer with hydroxyapatite or other fillers, molding, and subsequent thermal crosslinking. For instance, xylitol doped POC compositions can be formed by mixing pre-polymers with filler materials until a clay-life consistency is achieved, followed my molding into the desired shape and thermal crosslinking. Examples of filler materials include but are not limited to hydroxyapatite, B-tricalcium phosphate, pearl powder, octacalcium phosphate, etc.

Referring now to Tables 3 and 4, xylitol doped compositions were prepared both with a stoichiometric balance of —COOH and —OH functional groups among the monomers and with imbalanced ratios (favoring excess —OH groups with increased xylitol content). Excess —OH groups resulted in increased hydrogen bond interactions. In the case of the synthesized polymers, excess xylitol based —OH clusters led to areas of hydrogen bonding while still allowing crosslinking to proceed. Stoichiometrically balanced formulations led to polymers requiring extremely lengthy crosslinking times to achieve appreciable results. In a few cases where crosslinking was successful (N×1 and N×3), mechanics compared unfavorably with the corresponding unbalance formulation.

TABLE 3
Mole Ratio of Citric Acid: (Octanediol + Xylitol)
CXBE Formulations

| | Citric Acid | Xylitol | Octanediol |
|---|---|---|---|
| | (mols) | (mols) | (mols) |
| POC | 0.11 | 0 | 0.11 |
| X1 | 0.11 | 0.01 | 0.10 |
| X3 | 0.11 | 0.03 | 0.08 |
| X5 | 0.11 | 0.05 | 0.06 |
| X6 | 0.11 | 0.06 | 0.05 |
| X8 | 0.11 | 0.08 | 0.03 |

TABLE 4
1:1 Mole Ratio of —COOH:—OH
CXBE Formulations

| | Citric Acid | Xylitol | Octanediol |
|---|---|---|---|
| | (mols) | (mols) | (mols) |
| NX1 | 0.125 | 0.01 | 0.10 |
| NX2 | 0.155 | 0.03 | 0.08 |
| NX5 | 0.185 | 0.05 | 0.06 |
| NX6 | 0.20 | 0.06 | 0.05 |
| NX8 | 0.23 | 0.08 | 0.03 |

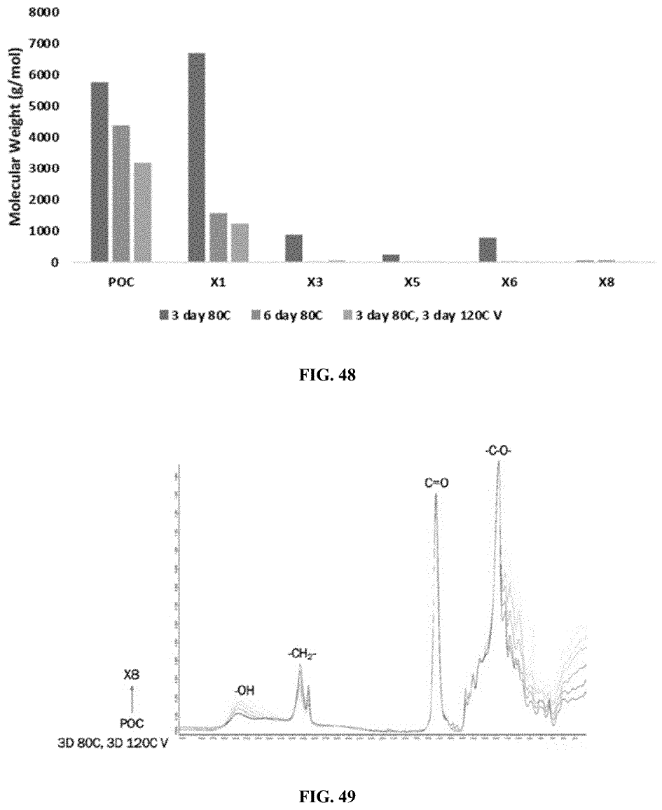

Referring to Table 5 and FIGS. 47 and 48, high strength, rapidly degradable polymer can be engineered by simultaneously increasing crosslinking density and hydrophilicity via xylitol incorporation. Incorporation of increasing amounts of xylitol leads to: decreased molecular weight, increasing polymer density, and vastly decreased molecular weight between crosslinks. Overall, results indicate formation of a highly branched and highly crosslinked polymer network, leading to increased mechanics while maintaining degradability due to the hydrophilic nature of xylitol.

TABLE 5
Molecular Weights of POC-Xylitol

| Xylitol (mols) | Mₙ | M_{w} | PDI |
|---|---|---|---|
| POC (control) | 1474 | 1624 | 1.10 |
| 0.01 | 1404 | 1543 | 1.10 |
| 0.03 | 1280 | 1377 | 1.08 |
| 0.05 | 1181 | 1257 | 1.06 |
| 0.06 | 1142 | 1206 | 1.06 |
| 0.08 | 1141 | 1198 | 1.05 |

Referring to FIG. 49, Fourier-transform infrared spectra of the compositions described above were obtained. An increased —OH signal was observed with increased levels of xylitol content within the polymer, indicating the formation of hydrogen bonds between polymer chains. This is further demonstrated by the broad slope of the —OH signal from 3300-3400. Such hydrogen bonds reinforce polymer mechanics.

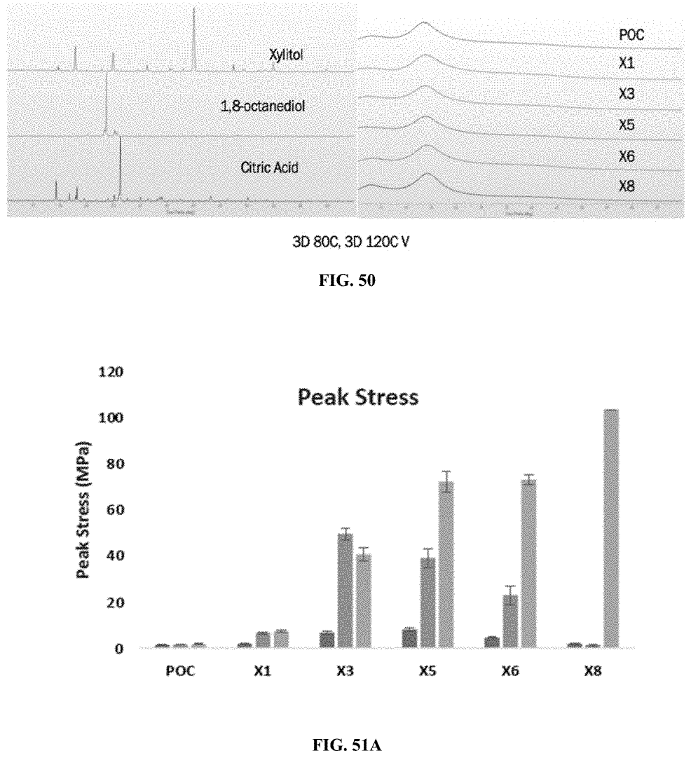

Referring to FIG. 50, x-ray diffraction spectra for the compositions described above were obtained. The spectra depict a lack of crystallinity of the polymers with increasing xylitol content.

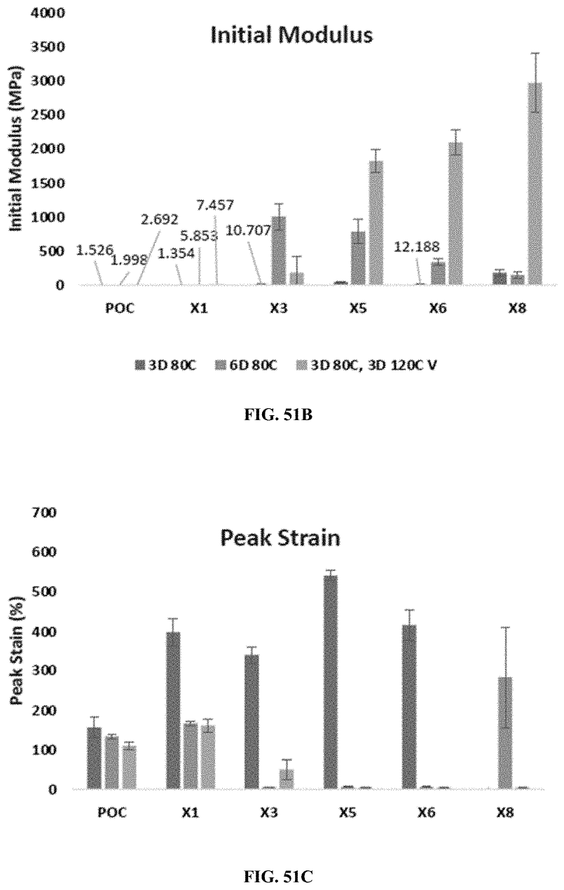

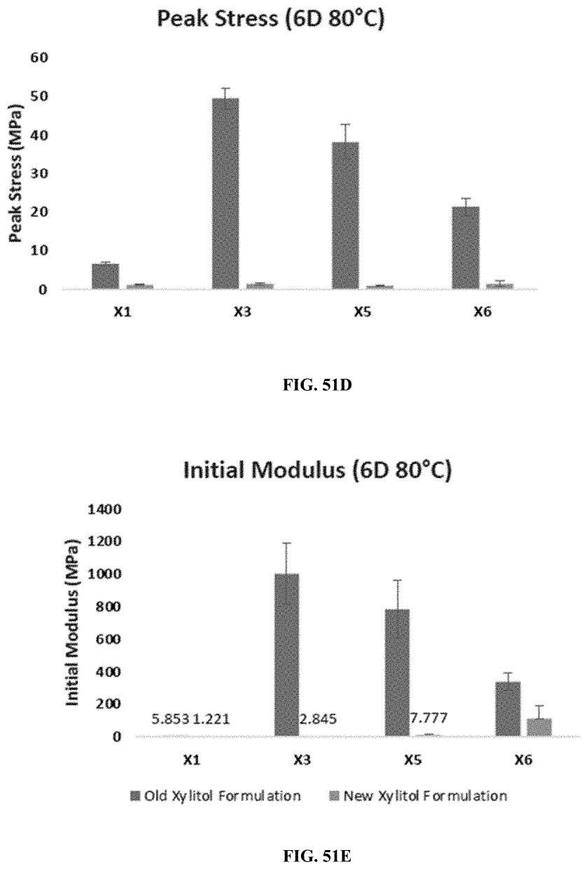

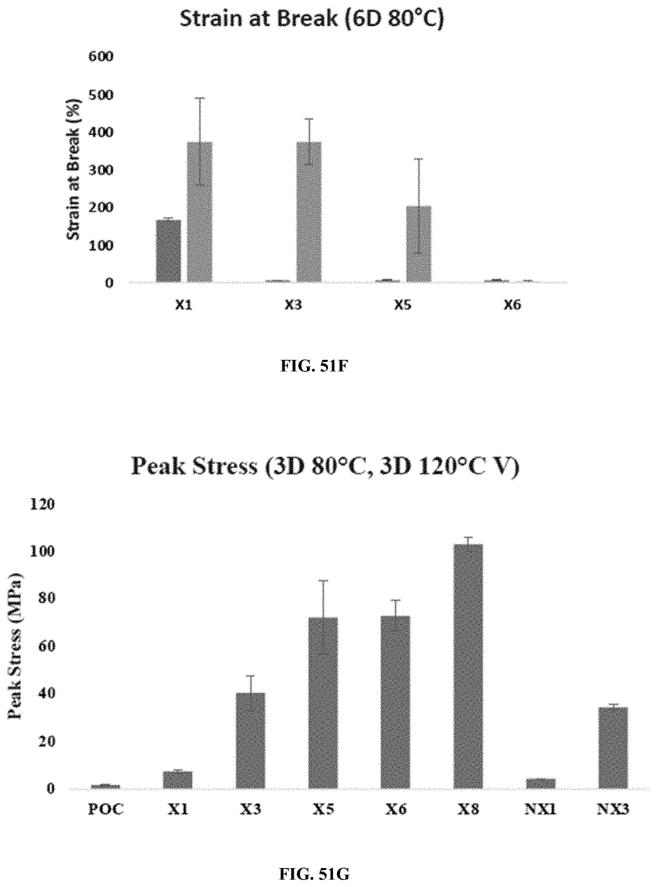

Referring to FIG. 51A-51G, polymer films were prepared from the compositions described above to analyze tensile film mechanics. Notably, formulations above X3 could not be crosslinked under the conditions used. The obtained measurements demonstrate the tunability of film mechanics in a manner that is capable of matching a range of biological tissues (such as skin, nerve, bone, etc.).

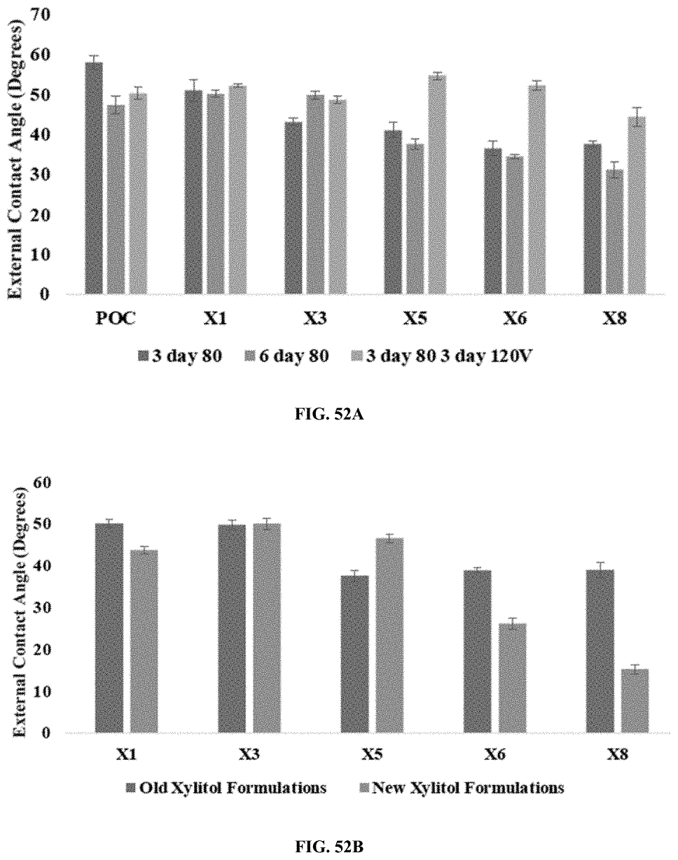

Referring to FIGS. 52A and 52B, the external contact angle for the compositions described above was measured. The observed contact angles demonstrate the hydrophilicity of the representative materials.

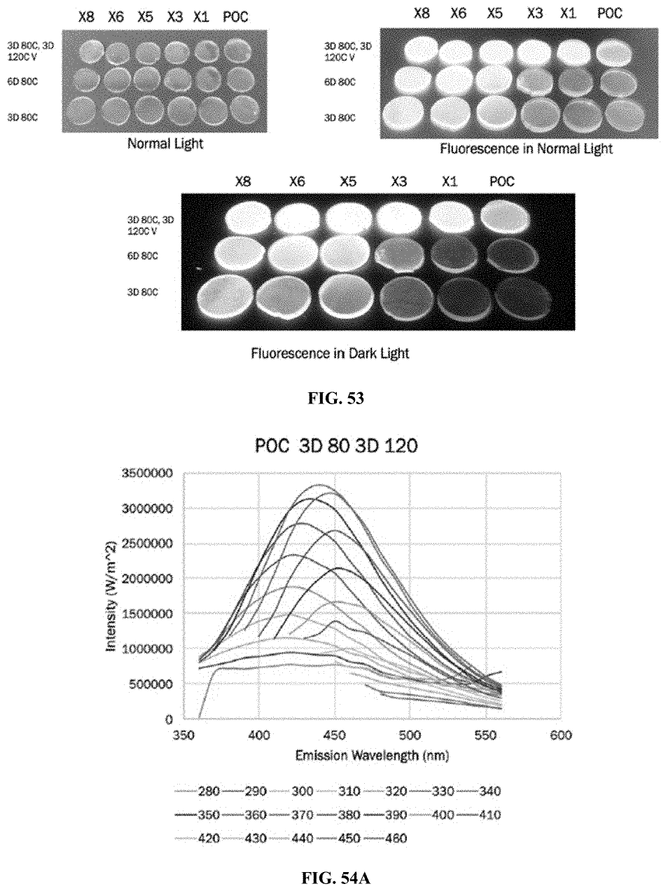

Referring to FIG. 53, the fluorescence of the prepared films was analyzed. Enhanced fluorescence was observed with increasing xylitol content. Increased branching and crosslink density with increasing xylitol content leads to increased hydrogen bond interactions between —OH and —C═O groups (pi-pi* and n-sigma* interactions), and thus increased fluorescence.

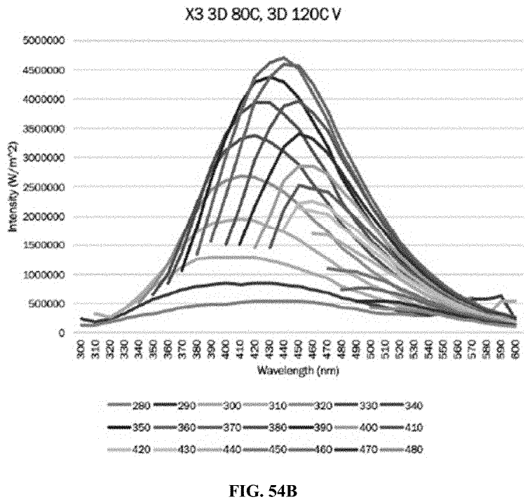

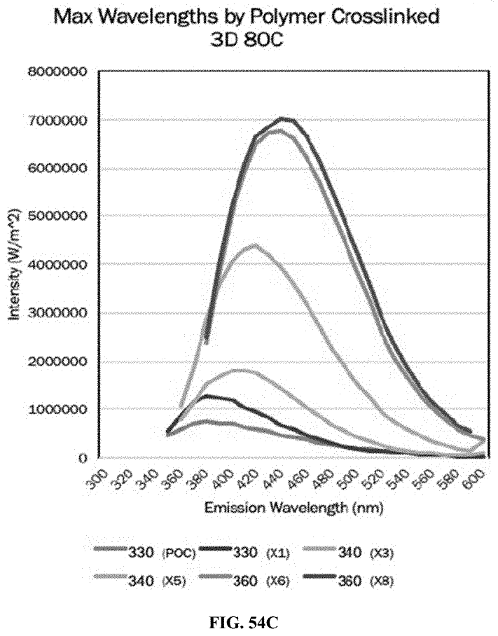

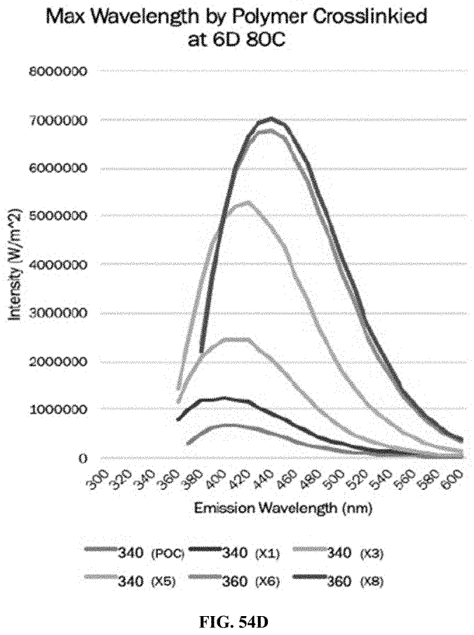

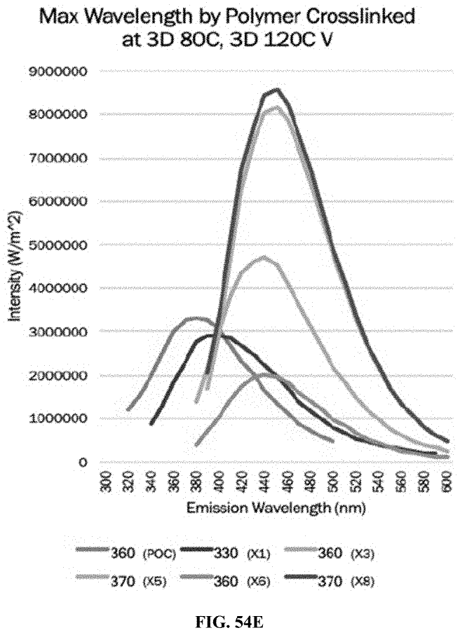

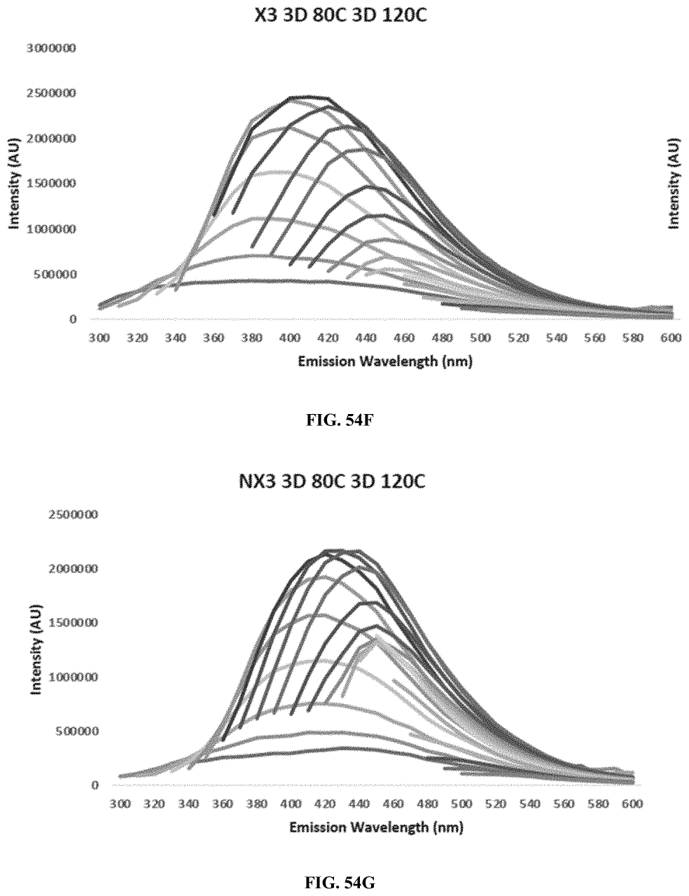

Referring to FIGS. 54A-54G, fluorescence emission spectra were obtained for the above-prepared compositions. These spectra show that the disclosed compositions may be useful for imaging and light delivery in vivo.

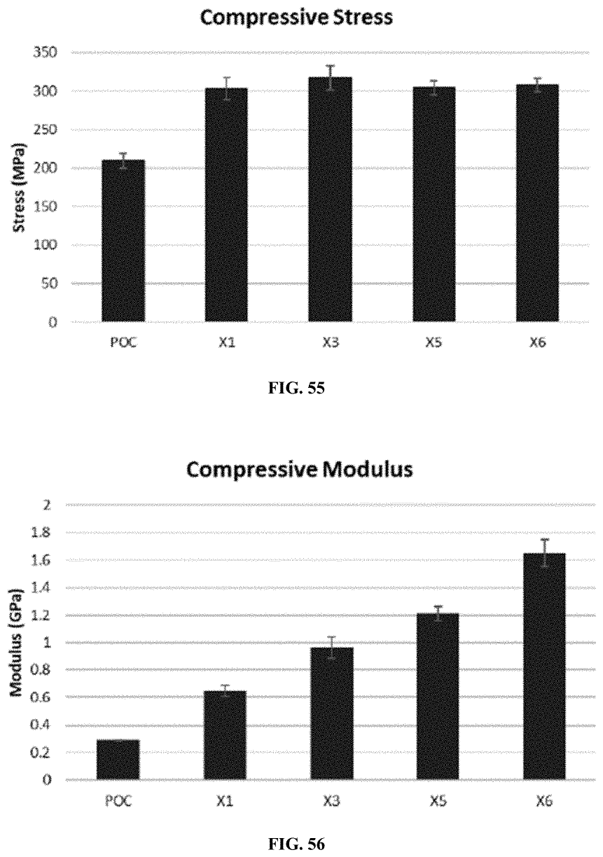

Referring now to FIG. 55, composites were prepared of the compositions described above and 60 weight percent hydroxyapatite (HA), and compressive mechanical properties of these compositions were analyzed. The obtained data demonstrate that uniform stress on the composites regardless of the xylitol content. Further, xylitol incorporation did not diminish the ability to incorporate HA, presumably due to the ability of xylitol to chelate ions.

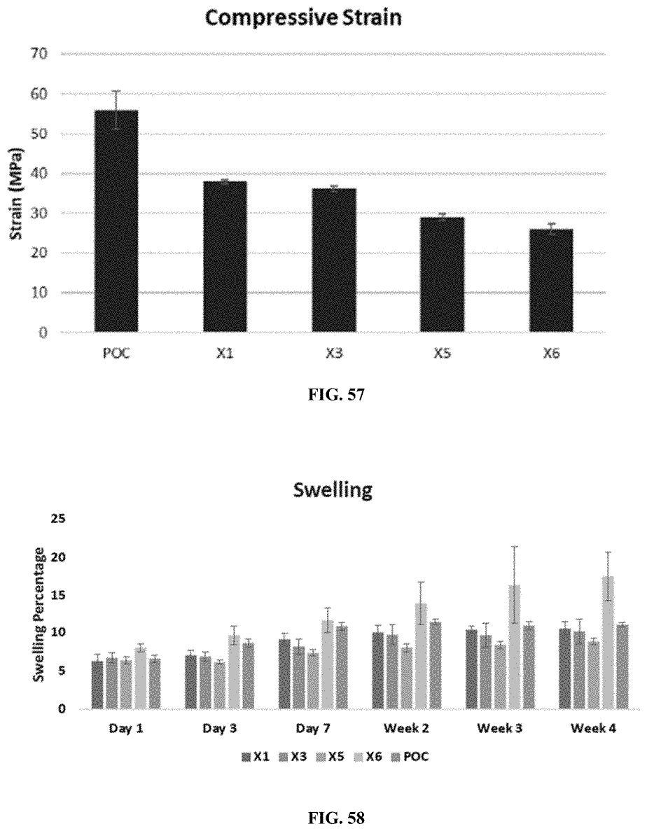

Referring now to FIG. 56, the compressive modulus of the prepared composites was analyzed. The values obtained were significantly enhanced compared to composites lacking xylitol. Measurements of compressive strain were also obtained (see FIG. 57).

Referring now to FIG. 58, the percentage of swelling for the prepared composites was analyzed. Composites containing xylitol were found to swell at the same rate as composites lacking xylitol despite the increased hydrophilic character of xylitol as a monomeric component.

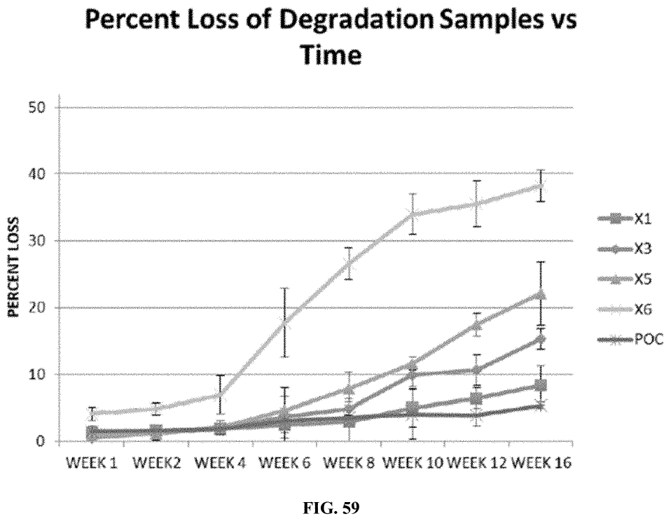

Referring now to FIG. 59, the degradation (in percent loss) of the disclosed compositions was analyzed over time. The compositions were found to have a tunable degradation rate of 5% to 40% over 16 weeks. Incorporating of higher amounts of xylitol led to complete loss of polymer weight (˜40%) in four months. Critically, the degradation rate can be tuned without negatively impacting or even significantly changing the initial mechanics of the composition.

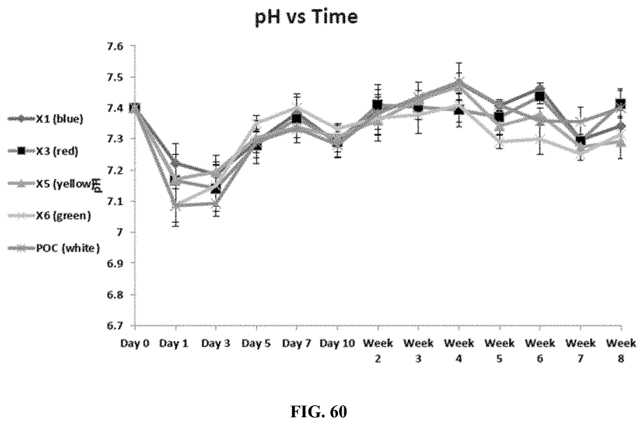

Referring now to FIG. 60, the pH of the composites over time was analyzed. A return to physiological pH (˜7.4) was observed within one week. An acute drop in pH can be associated with normal tissue healing while a prolonged acidic environment can be indicative of disease states or abnormal tissue healing; xylitol containing composites are capable of replicating a desired pH profile for the tissue environment.

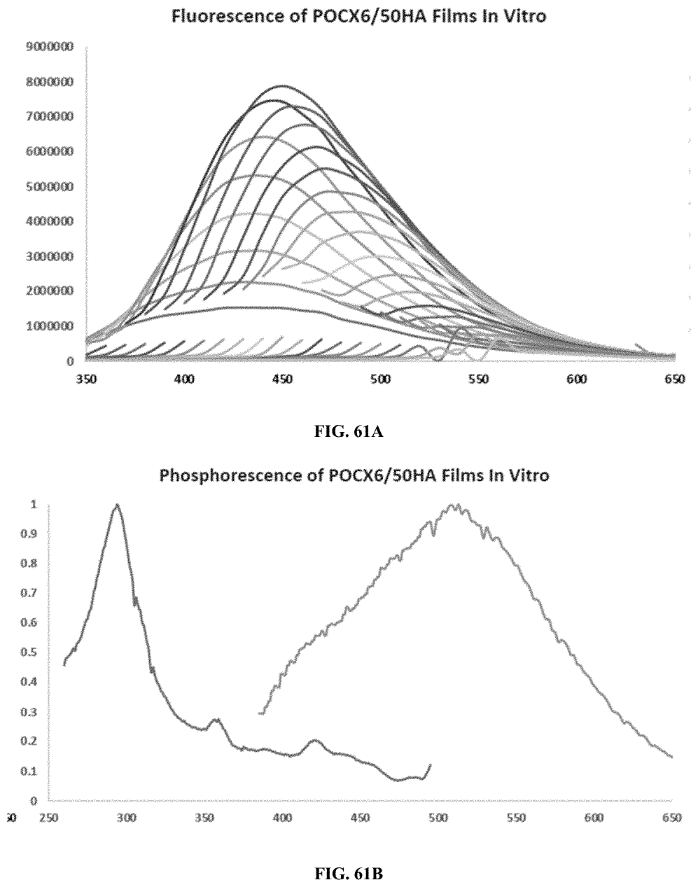

Referring to FIGS. 61A and 61B, fluorescence and room temperature phosphorescent spectra were obtained for the above described composites. The presence of room temperature phosphorescence demonstrates that these composites may be used in multiple imaging modalities. In particular, phosphorescence may be used preferentially in vivo to avoid the autofluorescence of biological tissues through the intrinsic delayed emission of phosphorescence versus fluorescence.

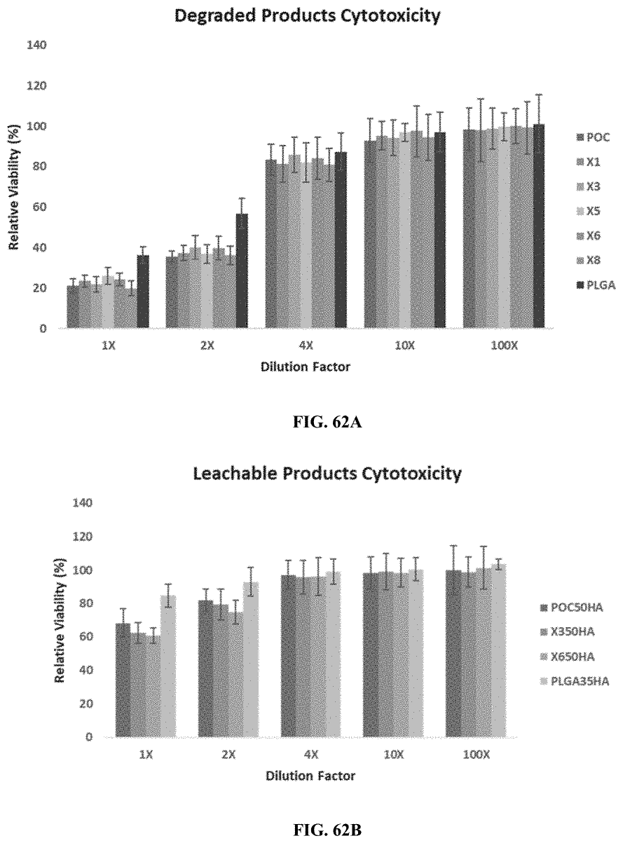

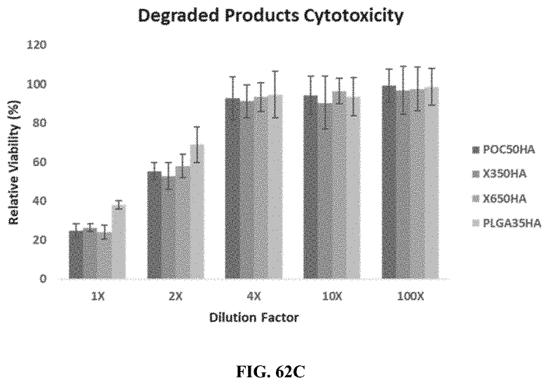

Referring to FIGS. 62A-62C, the in vitro cytotoxicity of the film degradation products and both the composite leachables and degradation products were evaluated against MG63 cells.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the device and methods of using and making the same disclosed herein have been discussed and illustrated, it is to be distinctly understood that the invention is not limited thereto but may be otherwise variously embodied and practiced within the scope of the following claims.

What is claimed is:

1. A catheter, comprising:
an elongated body defining one or more lumen;
the elongated body comprising a biodegradable crosslinked polymer.

2. The catheter recited in claim 1, wherein:
the crosslinked polymer is citrate/xylitol-based elastomer (CXBE).

3. The catheter recited in claim 1, further comprising:
a monitoring system comprising at least one moiety embedded within the elongated body, the moiety configured as a sensor.

4. The catheter recited in claim 1, further comprising:
a delivery system comprising at least one moiety embedded within the elongated body, the moiety configured to controllably release an agent encapsulated within the moiety.

5. The catheter recited in claim 1, further comprising:
a modulation system comprising at least one moiety embedded within the elongated body, the moiety configured to modulate flow of an agent through the one or more lumen or a portion of the elongated body.

6. The catheter recited in claim 1, further comprising:
a modulation system comprising at least one sensor embedded within the elongated body;
a delivery system comprising an encapsulation material that releases an agent upon degradation of the encapsulation or a shape memory material that releases an agent upon being activated; and
a modulation system that controls the degradation of the encapsulation material or controls the activation of the shape memory material.

7. The catheter recited in claim 1, further comprising:
a power harvester in electrical connection with any one or combination of:
a modulation system comprising at least one sensor embedded within the elongated body;
a delivery system comprising an encapsulation material that releases an agent upon degradation of the encapsulation or a shape memory material that releases an agent upon being activated; and
a modulation system that controls the degradation of the encapsulation material or controls the activation of the shape memory material.

8. The catheter recited in claim 7, further comprising:
a wireless module in electrical connection with the power harvester and in wireless communication with an operating module.

9. The catheter recited in claim 8, further comprising:
a pair of electrodes configured to generate electrical stimuli.

10. The catheter recited in claim 1, further comprising:
an anchoring mechanism configured to anchor the catheter to tissue.

11. The catheter recited in claim 10, wherein:
the anchoring mechanism includes any one or combination of: surface roughness of the catheter; nano- or micro-structures formed on a surface of the catheter; porous structures formed on a surface of the catheter; or adhesion moieties formed on a surface of the catheter.

12. The catheter recited in claim 2, wherein:
the CXBE is incorporated with epinephrine to generate epinephrine bearing CXBE (eCXBE).

13. The catheter recited in claim 10, wherein:
lidocaine is encapsulated within the eCBXE to generate eCBXE/lidocaine.

14. The catheter recited in claim 1, wherein:
the biodegradable crosslinked polymer contains a fluorescent polymer.

15. The catheter recited in claim 1, wherein:
the biodegradable crosslinked polymer has a differentiated crosslinked density through a cross-sectional portion of the elongated body;
the differentiated crosslinked density leading to differentiated swelling of the elongated body during water uptake.

16. A method of administering peripheral nerve block, the method comprising:
inserting a catheter in tissue of a patient;
allowing the catheter to swell so as to cause catheter anchorage to the tissue;
delivering agent via the catheter; and
allowing the catheter to biodegrade.

17. The method of administering peripheral nerve block recited in claim 16, wherein:
swelling is the only form of tissue anchorage for the catheter.

18. The method of administering peripheral nerve block recited in claim 16, wherein:
the catheter includes an elongated body defining one or more lumen;
swelling at or near the one or more lumen is less than swelling at an outer periphery of the elongated body.

19. The method of administering peripheral nerve block recited in claim 16, further comprising:
monitoring functionality and material degradation of the catheter via at least one moiety embedded within the catheter.

20. The method of administering peripheral nerve block recited in claim 16, further comprising:
delivering agent via at least one moiety embedded within the catheter.

21. The method of administering peripheral nerve block recited in claim 16, further comprising:
modulating flow of agent via at least one moiety embedded within the catheter.

22. The method of administering peripheral nerve block recited in claim 16, further comprising:
monitoring functionality and material degradation of the catheter via at least one monitoring moiety embedded within the catheter;
delivering agent via at least one delivering moiety embedded within the catheter;
modulating flow of agent via at least one modulating moiety embedded within the catheter;
providing electrical power to any one or combination of the monitoring moiety, the delivering moiety, or the modulating moiety via a power harvester embedded within the catheter.
